(19) United States
(12) Patent Application Publication
(10) Pub. No.: US 2026/0193649 A1
(43) Pub. Date: Jul. 9, 2026

(54) Branchpoint-Targeted Antisense Oligonucleotide for Restoring Pseudo-Exon Type Aberrant Splicing

(71) Applicant: Kyoto University, Kyoto (JP)

(72) Inventors: Masatoshi HAGIWARA, Kyoto (JP); Tomonari AWAYA, Kyoto (JP); Hiroaki OHARA, Kyoto (JP)

(73) Assignee: Kyoto University, Kyoto (JP)

(21) Appl. No.: 19/132,163

(22) PCT Filed: Nov. 22, 2023
(86) PCT No.: PCT/JP2023/041979

§ 371 Date: May 22, 2025

(30) Foreign Application Priority Data
Nov. 25, 2022 (JP) 2022-188542

(51) Int. Cl. C12N 15/113

(52) U.S. Cl. CPC C12N 15/113; C12N 2310/14; C12N 2310/321; C12N 2320/33

(57) ABSTRACT

In one aspect, the present disclosure provides an antisense oligonucleotide capable of correcting pseudo-exon-type aberrant splicing. In this aspect, the present disclosure provides an antisense oligonucleotide for correcting pseudo-exon-type aberrant splicing, having a base sequence capable of binding to a sequence that includes a branch point in pseudo-exon-type aberrant splicing.

### TECHNICAL FIELD

The present disclosure relates to a branch point-targeted antisense oligonucleotide for correcting pseudo-exon-type aberrant splicing.

### BACKGROUND ART

Splicing proceeds through a process in which, in RNA, a splice site at the end of an upstream exon binds to a branch point located downstream to temporarily form a lariat structure, followed by ligation after the lariat structure has been removed. The presence of aberrant branch points due to gene mutation alters the recognition of exon-intron boundaries, whereby proper splicing is disrupted. This leads to formation of incomplete mRNA in which sequences, such as introns, that are not normally used as exons (pseudo-exons) remain. As a result, translation of the mRNA into a protein stops in the middle of the process, and a necessary protein cannot be produced. Since this results in lack of the necessary protein, splicing defects can cause various diseases.

At present, more than fifteen nucleic acid therapeutics are approved worldwide, and five of them are approved also in Japan. For Duchenne muscular dystrophy, an exon skipping approach has been devised, in which an antisense oligonucleotide is used to induce exon 51 skipping. Further, exon trapping inhibitors applicable to Fukuyama congenital muscular dystrophy (FCMD) and using an antisense oligonucleotide are currently under investigator-initiated clinical trials (e.g., Patent Document 1 and Non-Patent Documents 1 and 2).

However, designing an antisense oligonucleotide requires an exhaustive search to select applicable genes and target sequences, and this requires a great deal of cost and effort. Moreover, such a great deal of cost and effort does not necessarily lead to successful designing of a sequence suitable for desired skipping of an exon. They have been a bottleneck in the development of antisense oligonucleotides.

### PRIOR ART DOCUMENTS

### Patent Document

Patent Document 1: JP 2013-216595A

### Non-Patent Documents

Non-Patent Document 1: Kobayashi, K., et al. (1998). Nature 394, 388-392.
Non-Patent Document 2: Taniguchi-Ikeda, M., et al. (2011). Nature 478, 127-131.

### DISCLOSURE OF INVENTION

### Problem to be Solved by the Invention

The present disclosure provides a novel antisense oligonucleotide capable of correcting pseudo-exon-type aberrant splicing.

### Means for Solving Problem

One aspect of the present disclosure relates to an antisense oligonucleotide for correcting pseudo-exon-type aberrant splicing, having a base sequence capable of binding to a sequence that includes a branch point in pseudo-exon-type aberrant splicing.

Another aspect of the present disclosure relates to an antisense oligonucleotide including a base sequence capable of binding to a target sequence consisting of 10 to 50 successive bases in a region selected from the group consisting of regions (1) to (40) to be described below, wherein the base sequence includes a branch point in pseudo-exon-type aberrant splicing.

Still another aspect of the present disclosure relates to a pharmaceutical composition containing the antisense oligonucleotide of the present disclosure.

Yet another aspect of the present disclosure relates to a method for producing an antisense oligonucleotide for correcting pseudo-exon-type aberrant splicing, comprising producing a base sequence that is complementary to a target sequence that includes a branch point in pseudo-exon-type aberrant splicing and consists of 10 to 50 bases.

### Effects of the Invention

According to one aspect of the present disclosure, a novel antisense oligonucleotide capable of correcting pseudo-exon-type aberrant splicing can be provided.

### DESCRIPTION OF THE INVENTION

An antisense oligonucleotide is typically designed so as to target, in a sequence that regulates exon recognition, two splice sites located upstream and downstream and splicing enhancers present inside and outside of an exon. However, this method has a problem in that, since the sequences of the splicing enhancers are not clear, it is difficult to identify in which part of the gene a target sequence appears. In order to solve this problem, a method of designing antisense oligonucleotides so as to comprehensively cover a wide region centered on the splice sites and evaluating the antisense oligonucleotides has been commonly employed. However, such a method also has problems in that it requires an exhaustive search over a wide region and thus requires a great deal of cost and effort, and that such a great deal of cost and effort does not necessarily lead to successful designing of a suitable sequence.

In light of these problems, the inventors of the present invention conducted studies to find a method that enables efficient designing of antisense oligonucleotides effective against a number of applicable diseases. In the course of these studies, the inventors focused on a branch point (BP). It is known that the branch point corresponds to any of adenine bases located 10 to 120 bases upstream of the 3′-splice site of an intron. Accordingly, it was expected that identification of the branch point would be relatively easy. Further, even when there is a plurality of candidate branch points, they can be evaluated easily because a region including the plurality of candidate branch points can be covered by designing several antisense oligonucleotides.

Based on these findings, the inventors made an attempt to design a branch point-targeted antisense oligonucleotide in order to correct pseudo-exon-type aberrant splicing in an applicable disease Fukuyama congenital muscular dystrophy, which is prevalent in Japan. As a result, antisense oligonucleotides that exhibit high pseudo-exon skipping efficiency and favorable restoration of target gene functions could be obtained efficiently in a very simple manner.

Branch points have alternative sites. Thus, there was a concern that, even if a particular branch point is inhibited, another branch point may act in a compensatory manner. However, such a problem did not occur.

In one or more embodiments, a branch point is a specific nucleotide (mainly adenine base) that is located upstream of the 3′-splice site of an intron and serves as a starting point of splicing. In other words, the branch point is present in a non-coding region. Upon binding of U2 snRNA to an adenine base as the branch point, a hydroxy group at the 2′-position of the adenine base attacks and cleaves the 5′-splice site at the 5′end of the intron. The thus-cleaved portion binds to the branch point (adenine base) to form a lariat structure, and the 3′-splice site at the 3′end of the intron is cleaved. As a result, the intron is cleaved off and then degraded.

### Antisense Oligonucleotide

One aspect of the present disclosure relates to an antisense oligonucleotide for correcting pseudo-exon-type aberrant splicing, having a base sequence capable of binding to a sequence including a branch point in pseudo-exon-type aberrant splicing. In one or more embodiments, the antisense oligonucleotide of the present disclosure has a base sequence capable of binding to a sequence including a branch point that serves as a starting point of pseudo-exon-type aberrant splicing. Accordingly, by binding the antisense oligonucleotide of the present disclosure to the sequence including the branch point, binding of U2 snRNP to the branch point is suppressed, whereby the occurrence of pseudo-exon-type aberrant splicing can be corrected. In addition, in one or more embodiments, the antisense oligonucleotide of the present disclosure can increase the production of correctly spliced transcripts and thus can treat diseases caused by pseudo-exon-type aberrant splicing.

In one or more embodiments, pseudo-exon-type aberrant splicing is aberrant splicing resulting from recognition of a non-coding region as a pseudo-exon due to mutation in an intron region.

In one or more embodiments, the length of the antisense oligonucleotide of the present disclosure is 10 to 50 bases. In one or more embodiments, the length of the antisense oligonucleotide of the present disclosure is 11 bases or more, 12 bases or more, 13 bases or more, 14 bases or more, or 15 bases or more. In one or more embodiments, the length of the antisense oligonucleotide of the present disclosure is 45 bases or less, 40 bases or less, 39 bases or less, 38 bases or less, 37 bases or less, 36 bases or less, 35 bases or less, 34 bases or less, 33 bases or less, 32 bases or less, 30 bases or less, 29 bases or less, 28 bases or less, 27 bases or less, 26 bases or less, or 25 bases or less.

The antisense oligonucleotide of the present disclosure has a base sequence capable of binding to a sequence including a branch point that serves as a starting point of pseudo-exon-type aberrant splicing. In one or more embodiments, the antisense oligonucleotide of the present disclosure may include a base capable of binding to the above-described branch point, and there is no particular limitation on the position of the base capable of binding to the branch point. In one or more embodiments, the base capable of binding to the branch point may be the second base, third base, fourth base, fifth base, sixth base, or any subsequent base from the 5′end of the antisense oligonucleotide of the present disclosure. In one or more embodiments, the base capable of binding to the branch point may be the second base, third base, fourth base, fifth base, sixth base, or any subsequent base from the 3′end of the antisense oligonucleotide of the present disclosure.

In one or more embodiments, the antisense oligonucleotide of the present disclosure can reduce and/or suppress the occurrence of recognition of a non-coding region as a pseudo-exon by binding to the sequence including the branch point, thereby correcting pseudo-exon-type aberrant splicing and thus allowing normal splicing. In one or more embodiments, the antisense oligonucleotide of the present disclosure can suppress aberrant splicing involving pseudo-exon formation with an efficiency of 10% or more, 20% or more, 30% or more, 40% or more, 50% or more, 60% or more, 70% or more, 80% or more, or 90% or more.

In the present disclosure, “correcting or suppressing pseudo-exon-type aberrant splicing” means that, in one or more embodiments, in a patient having a disease caused by pseudo-exon-type aberrant splicing, the expression level of aberrant mRNA and/or the expression level of aberrant transcripts produced by the pseudo-exon-type aberrant splicing is suppressed. Suppression of the expression level means, in one or more embodiments, the expression level is reduced by 20% or more, 25% or more, 30% or more, 40% or more, 45% or more, or 50% or more, as compared with the expression level before administering the antisense oligonucleotide of the present disclosure.

In the present disclosure, “increasing the production of correctly spliced transcripts” means that, in one or more embodiments, in a patient having a disease caused by pseudo-exon-type aberrant splicing or in a sample (specimen) derived from the patient, the amount of production (expression level) of the correctly spliced transcripts is increased by 20% or more, 25% or more, 30% or more, 40% or more, 45% or more, or 50% or more, as compared with the expression level before administering the antisense oligonucleotide of the present disclosure. In one or more embodiments, examples of the sample (specimen) derived from the patient include cells, tissue, and organs collected from the patient.

In one or more embodiments, examples of the pseudo-exon-type aberrant splicing in the present disclosure include pseudo-exon-type splicing defects observed in genetic diseases, cancers, etc.

In one or more embodiments, examples of the disease caused by pseudo-exon-type aberrant splicing include: Stargardt disease, ABCA4; hyperinsulinemic hypoglycemia, familial, ABCC8, HADH; familial adenomatous polyposis, APC ataxia telangiectasia, ATM; breast-ovarian cancer, familial, BRCA1, BRCA2; breast cancer, early-onset, susceptibility to, BRIP1; Leber congenital amaurosis, CEP290, RPGRIP1; cystic fibrosis, CFTR; Usher syndrome, CLRN1, USH2A; achromatopsia, CNGB3; Ullrich congenital muscular dystrophy, COL6A1; muscular dystrophy, limb-girdle, autosomal recessive, DYSF; Fukuyama congenital muscular dystrophy, FKTN; Pompe disease, GAA; galactosemia, GALT; Laron dwarfism, GHR; Fabry disease, GLA; beta-thalassemia, EBB; hypercholesterolemia, familial, LDLR; familial hypertrophic cardiomyopathy, MYBPC3; autosomal recessive polycystic kidney disease, PKHD1; Gitelman syndrome, SLC12A3; and Werner syndrome, WRN (Alphanumeric characters following each disease name indicate the causative gene(s) for the disease).

Examples of the mutation causing pseudo-exon-type splicing defects include, in one or more embodiments, mutations in the causative genes for the above-described diseases. Examples of the causative genes for the above-described diseases include, in one or more embodiments, the ABCA4 gene, ABCC8 gene, APC gene, ATM gene, BRCA1 gene, BRCA2 gene, BRIP1 gene, CEP290 gene, CFTR gene, CLRN1 gene, CNGB3 gene, COL6A1 gene, DYSF gene, FKTN gene, GAA gene, GALT gene, GHR gene, GLA gene, HADH gene, EBB gene, LDLR gene, MYBPC3 gene, PKHD1 gene, RPGRIP1 gene, SLC12A3 gene, USH2A gene, and WRN gene.

In one or more embodiments, mutations causing the pseudo-exon-type splicing defects include the following mutations (variants) in the above-described genes. In one or more embodiments, the antisense oligonucleotide of the present disclosure has activity capable of suppressing and/or correcting pseudo-exon-type aberrant splicing caused by the following mutations (variants) in the above-described genes. In one or more embodiments, whether the pseudo-exon-type aberrant splicing is suppressed and/or corrected can be determined by examining the base sequences of splicing products (transcripts). The following mutations are represented according to the notation system established by the Human Genome Variation Society with respect to base sequences identified by Accession IDs in the RefSeq database (NCBI Reference Sequence Database, https://wwwncbi.nhm.nih.gov/refseq/) provided by NCBI (National Center for Biotechnology Information, https://wwwncbi.nlm.nih.gov/) in the United States.
NC_000001.11:g.94084225G>Amutation in theABCA4 gene;
NC_000001.11:g.94081224C>G mutation in the ABCA4 gene;
NC_000001.11:g.94062142G>C mutation in the ABCA4 gene;
NC_000001.11:g.94028345T>C mutation in theABCA4 gene;
NC_000001.11:g.94027444C>T mutation in theABCA4 gene;
NC_000011.11:g.17444325T>C mutation in theABCC8 gene;
NC_000005.11:g.112790640T>G mutation in the APC gene;
NC_000005.11:g.112822720A>G mutation in the APC gene;
NC_000005.11:g.112822722C>T mutation in the APC gene;
NC_000005.11:g.112822726A>T mutation in the APC gene;
NC_000005.11:g.112822734G>A mutation in the APC gene;
NC_000005.11:g.112779849G>A mutation in the APC gene;
NC_000011.11:g.108270483_108270486del mutation in the ATM gene;
NC_000011.11:g.108309110A>G mutation in the ATM gene;
NC_000017.11:g.43086839G>Amutation in the BRCA1 gene;
NC_000013.11:g.32345247T>G mutation in the BRCA2 gene;
NC_000017.11:g.61781503T>A mutation in the BRIP1 gene;
NC_000012.11:g.88101183T>C mutation in the CEP290 gene;
NC_000007.11:g.117578327A>G mutation in the CFTR gene;
NC_000007.11:g.117589467A>G mutation in the CFTR gene;
NC_000007.11:g.117639961C>T mutation in the CFTR gene;
NC_000003.11:g.150942410A>C mutation in the CLRN1 gene;
NC_000008.11:g.86605416C>T mutation in the CNGB3 gene;
NC_000021.11:g.45989967C>T mutation in the COL6A1 gene;
NC_000002.11:g.71661900G>T mutation in the DYSF gene;
NC_000009.11:g.105606576G>T mutation in the FKTN gene;
NC_000017.11:g.80104542T>G mutation in the GAA gene;
NC_000009.11:g.34649954A>G mutation in the GALT gene;
NC_000005.11:g.42700794A>G mutation in the GHR gene;
NC_000023.11:g.101399747C>T mutation in the GLA gene;
NC_000004.11:g.108023948A>G mutation in the HADH gene;
NC_000011.11:g.5225872A>C mutation in the EBB gene;
NC_000011.11:g.5225832G>C mutation in the HBB gene;
NC_000011.11:g.5225923G>Amutation in the EBB gene;
NC_000019.11:g.11120625G>T mutation in the LDLR gene;
NC_000019.11:g.11122956G>A mutation in the LDLR gene;
NC_000011.11:g.47343314C>T mutation in the MYBPC3 gene;
NC_000006.11:g.51882440T>C mutation in the PKHD1 gene;
NC_000014.11:g.21321131T>G mutation in the RPGRIP1 gene;
NC_000016.11:g.56883858C>T mutation in the SLC12A3 gene;
NC_000016.11:g.56893307C>T mutation in the SLC12A3 gene;
NC_000001.11:g.215891198T>C mutation in the USH2A gene;
NC_000001.11:g.215794441T>C mutation in the USH2A gene; and
NC_000008.11:g.31108591A>G mutation in the WRN gene.

FIG. 1 shows the names of diseases that can be target diseases of the antisense oligonucleotide of the present disclosure, as well as causative genes, mutations, ClinVar registration numbers, dbSNP registration numbers, and examples of branch points corresponding to the respective diseases. In one or more embodiments, the antisense oligonucleotide of the present disclosure is capable of correcting pseudo-exon-type aberrant splicing that can occur in patients having the diseases shown in FIG. 1 (pseudo-exon-type aberrant splicing in the genes listed in FIG. 1).

SEQ ID NO: 1 is the base sequence of the ABCA4 gene, and represents the reverse complement of the base sequence consisting of 93992834th to 94121148th bases in the base sequence of human chromosome 1 in a GRCh38/hg38 human reference genome list (NC_000001.11 (93992834.94121148, complement)).

SEQ ID NO: 2 is the base sequence of the ABCC8 gene, and represents the reverse complement of the base sequence consisting of 17392498th to 17476845th bases in the base sequence of human chromosome 11 in the GRCh38/hg38 human reference genome list (NC_000011.10 (17392498.17476845, complement)).

SEQ ID NO: 3 is the base sequence of the APC gene, and represents the base sequence consisting of 112707498th to 112846239th bases in the base sequence of human chromosome 5 in the GRCh38/hg38 human reference genome list (NC_000005.10 (112707498.112846239)).

SEQ ID NO: 4 is the base sequence of the ATM gene, and represents the base sequence consisting of 108223067th to 108369102nd bases in the base sequence of human chromosome 11 in the GRCh38/hg38 human reference genome list (NC_000011.10 (108223067.108369102)).

SEQ ID NO: 5 is the base sequence of the BRCA1 gene, and represents the reverse complement of the base sequence consisting of 43044295th to 43125364th bases in the base sequence of human chromosome 17 in the GRCh38/hg38 human reference genome list (NC_000017.11 (43044295.43125364, complement)).

SEQ ID NO: 6 is the base sequence of the BRCA2 gene, and represents the base sequence consisting of 32315508th to 32400268th bases in the base sequence of human chromosome 13 in the GRCh38/hg38 human reference genome list (NC_000013.11 (32315508.32400268)).

SEQ ID NO: 7 is the base sequence of the BRIP1 gene, and represents the reverse complement of the base sequence consisting of 61/679,139th to 61/863,528th bases in the base sequence of human chromosome 17 in the GRCh38/hg38 human reference genome list (NC_000017.11 (61679139.61863528, complement)).

SEQ ID NO: 8 is the base sequence of the CEP290 gene, and represents the reverse complement of the base sequence consisting of88049016th to 88142088th bases in the base sequence of human chromosome 12 in the GRCh38/hg38 human reference genome list (NC_000012.12 (88049016.88142088, complement)).

SEQ ID NO: 9 is the base sequence of the CFTR gene, and represents the base sequence consisting of 117480025th to 117668665th bases in the base sequence of human chromosome 7 in the GRCh38/hg38 human reference genome list (NC_000007.14 (117480025.117668665)).

SEQ ID NO: 10 is the base sequence of the CLRN1 gene, and represents the reverse complement of the base sequence consisting of 150926163rd to 150972999th bases in the base sequence of human chromosome 3 in the GRCh38/hg38 human reference genome list (NC_000003.12 (150926163.150972999, complement)).

SEQ ID NO: 11 is the base sequence of the CNGB3 gene, and represents the reverse complement of the base sequence consisting of86574179th to 86743634th bases in the base sequence of human chromosome 8 in the GRCh38/hg38 human reference genome list (NC_000008.11 (86574179.86743634, complement)).

SEQ ID NO: 12 is the base sequence of the COL6A1 gene, and represents the base sequence consisting of 45981770th to 46005048th bases in the base sequence of human chromosome 21 in the GRCh38/hg38 human reference genome list (NC_000021.9 (45981770.46005048)).

SEQ ID NO: 13 is the base sequence of the DYSF gene, and represents the base sequence consisting of 71453561st to 71686763rd bases in the base sequence of human chromosome 2 in the GRCh38/hg38 human reference genome list (NC_000002.12 (71453561.71686763)).

SEQ ID NO: 14 is the base sequence of the FKTN gene, and represents the base sequence consisting of 105558130th to 105641118th bases in the base sequence of human chromosome 9 in the GRCh38/hg38 human reference genome list (NC_000009.12 (105558130.105641118)).

SEQ ID NO: 15 is the base sequence of the GAA gene, and represents the base sequence consisting of80101581st to 80119881st bases in the base sequence of human chromosome 17 in the GRCh38/hg38 human reference genome list (NC_000017.11 (80101581.80119881)).

SEQ ID NO: 16 is the base sequence of the GALT gene, and represents the base sequence consisting of 34646675th to 34651035th bases in the base sequence of human chromosome 9 in the GRCh38/hg38 human reference genome list (NC_000009.12 (34646675.34651035)).

SEQ ID NO: 17 is the base sequence of the GHR gene, and represents the base sequence consisting of 42423439th to 42721878th bases in the base sequence of human chromosome 5 in the GRCh38/hg38 human reference genome list (NC_000005.10 (42423439.42721878)).

SEQ ID NO: 18 is the base sequence of the GLA gene, and represents the reverse complement of the base sequence consisting of 101397803rd to 101407925th bases in the base sequence of human chromosome X in the GRCh38/hg38 human reference genome list (NC_000023.11 (101397803.101407925, complement)).

SEQ ID NO: 19 is the base sequence of the HADH gene, and represents the base sequence consisting of 107989889th to 108035171st bases in the base sequence of human chromosome 4 in the GRCh38/hg38 human reference genome list (NC_000004.12 (107989889.108035171)).

SEQ ID NO: 20 is the base sequence of the HBB gene, and represents the reverse complement of the base sequence consisting of 5225464th to 5227071st bases in the base sequence of human chromosome 11 in the GRCh38/hg38 human reference genome list (NC_000011.10 (5225464.5227071, complement)).

SEQ ID NO: 21 is the base sequence of the LDLR gene, and represents the base sequence consisting of 11089463rd to 11133820th bases in the base sequence of human chromosome 19 in the GRCh38/hg38 human reference genome list (NC_000019.10 (11089463.11133820)).

SEQ ID NO: 22 is the base sequence of the MYBPC3 gene, and represents the reverse complement of the base sequence consisting of 47331406th to 47352702nd bases in the base sequence of human chromosome 11 in the GRCh38/hg38 human reference genome list (NC_000011.10 (47331406.47352702, complement)).

SEQ ID NO: 23 is the base sequence of the PKHD1 gene, and represents the reverse complement of the base sequence consisting of 51615299th to 52087615th bases in the base sequence of human chromosome 6 in the GRCh38/hg38 human reference genome list (NC_000006.12 (51615299.52087615, complement)).

SEQ ID NO: 24 is the base sequence of the RPGRIP1 gene, and represents the base sequence consisting of 21280083rd to 21351301st bases in the base sequence of human chromosome 14 in the GRCh38/hg38 human reference genome list (NC_000014.9 (21280083.21351301)).

SEQ ID NO: 25 is the base sequence of the SLC12A3 gene, and represents the base sequence consisting of 56865207th to 56915850th bases in the base sequence of human chromosome 16 in the GRCh38/hg38 human reference genome list (NC_000016.10 (56865207.56915850)).

SEQ ID NO: 26 is the base sequence of the USH2A gene, and represents the reverse complement of the base sequence consisting of 215622891st to 216423448th bases in the base sequence of human chromosome 1 in the GRCh38/hg38 human reference genome list (NC_000001.11 (215622891.216423448, complement)).

SEQ ID NO: 27 is the base sequence of the WRN gene, and represents the base sequence consisting of 31033810th to 31176138th bases in the base sequence of human chromosome 8 in the GRCh38/hg38 human reference genome list (NC_000008.11 (31033810.31176138)).

In the present disclosure, “GRCh38/hg38” refers to an assembly of human genome data (RefSeq assembly accession: GRCh38.p14 (GCF_000001405.40)) registered in https://www.ncbi.nhm.nih.gov/and the like.

In one or more embodiments, the branch point in the present disclosure is an adenine base located in a region from 10 to 120 bases upstream of the 3′-splice site of an intron. In one or more embodiments, the region where the adenine base as the branch point is present may be a region from 11, 12, 13, 14, 15, 16, 17, 18, 19, 20, or 21 bases to 35, 40, 41, 42, 43, 44, 45, 50, 55, 65, 70, 80, 90, or 100 bases upstream of the 3′-splice site of the intron.

In one or more embodiments, the branch point can be predicted using SVM-BP finder (http://regulatorygenomics.udf.edu/Software/SVM_BP/).

In one or more embodiments, the branch point in the present disclosure may be an adenine base present in a region where branch points that may be involved in pseudo-exon-type aberrant splicing in the above-described causative genes (regions (1) to (40) to be described below: base sequences represented by SEQ ID NOs: 28 to 67). In one or more embodiments, examples of the branch point of the present disclosure include adenine bases to be described below. In the following table column entitled “Location in GRCh38/hg38 human reference genome”, “chr**” indicates the chromosomal location.

TABLE 1
The branch point that can be involved in pseudo-exon-
type aberrant splicing caused by the mutation (chr1:
94084225G > A) in the ABCA4 gene (SEQ ID NO: 1):

| | Location in the | Location in |
|---|---|---|
| Location in GRCh38/hg38 | causative gene | the regen |
| human reference genome | (SEQ ID NO: 1) | (SEQ ID NO: 28) |
| chr1: 94084303 | 36846 | 37 |
| chr1: 94084298 | 36851 | 42 |
| chr1: 94084288 | 36861 | 52 |
| chr1: 94084274 | 36875 | 66 |
| chr1: 94084253 | 36896 | 87 |
| chr1: 94084247 | 36902 | 93 |
| chr1: 94084240 | 36909 | 100 |

The branch point that can be involved in pseudo-exon-
type aberrant splicing caused by the mutation (chr1:
94081224C > G) in the ABCA4 gene (SEQ ID NO: 1):

| | Location in the | Location in |
|---|---|---|
| Location in GRCh38/hg38 | causative gene | the regen |
| human reference genome | (SEQ ID NO: 1) | (SEQ ID NO: 29) |
| chr1: 94081310 | 39839 | 32 |
| chr1: 94081305 | 39844 | 37 |
| chr1: 94081259 | 39890 | 83 |
| chr1: 94081251 | 39898 | 91 |

The branch point that can be involved in pseudo-exon-
type aberrant splicing caused by the mutation (chr1:
94062142G > C) in the ABCA4 gene (SEQ ID NO: 1):

| | Location in the | Location in |
|---|---|---|
| Location in GRCh38/hg38 | causative gene | the regen |
| human reference genome | (SEQ ID NO: 1) | (SEQ ID NO: 30) |
| chr1: 94062241 | 58908 | 61 |
| chr1: 94062224 | 58925 | 78 |
| chr1: 94062213 | 58936 | 89 |
| chr1: 94062206 | 58943 | 96 |

The branch point that can be involved in pseudo-exon-
type aberrant splicing caused by the mutation (chr1:
94028345T > C) in the ABCA4 gene (SEQ ID NO: 1):

| | Location in the | Location in |
|---|---|---|
| Location in GRCh38/hg38 | causative gene | the regen |
| human reference genome | (SEQ ID NO: 1) | (SEQ ID NO: 31) |
| chr1: 94028507 | 92642 | 26 |
| chr1: 94028506 | 92643 | 27 |
| chr1: 94028498 | 92651 | 35 |
| chr1: 94028497 | 92652 | 36 |
| chr1: 94028494 | 92655 | 39 |
| chr1: 94028493 | 92656 | 40 |
| chr1: 94028466 | 92683 | 67 |

The branch point that can be involved in pseudo-exon-
type aberrant splicing caused by the mutation (chr1:
94028345T > C) in the ABCA4 gene (SEQ ID NO: 1):

| | Location in the | Location in |
|---|---|---|
| Location in GRCh38/hg38 | causative gene | the regen |
| human reference genome | (SEQ ID NO: 1) | (SEQ ID NO: 32) |
| chr1: 94028544 | 92605 | 33 |
| chr1: 94028534 | 92615 | 43 |
| chr1: 94028518 | 92631 | 59 |
| chr1: 94028510 | 92639 | 67 |
| chr1: 94028507 | 92642 | 70 |
| chr1: 94028506 | 92643 | 71 |
| chr1: 94028498 | 92651 | 79 |
| chr1: 94028497 | 92652 | 80 |
| chr1: 94028494 | 92655 | 83 |
| chr1: 94028493 | 92656 | 84 |

The branch point that can be involved in pseudo-exon-
type aberrant splicing caused by the mutation (chr1:
94027444C > T) in the ABCA4 gene (SEQ ID NO: 1):

| | Location in the | Location in |
|---|---|---|
| Location in GRCh38/hg38 | causative gene | the regen |
| human reference genome | (SEQ ID NO: 1) | (SEQ ID NO: 33) |
| chr1: 94027614 | 93535 | 61 |
| chr1: 94027606 | 93543 | 69 |
| chr1: 94027592 | 93557 | 83 |
| chr1: 94027583 | 93566 | 92 |

The branch point that can be involved in pseudo-exon-
type aberrant splicing caused by the mutation (chr11:
17444325T > C) in the ABCC8 gene (SEQ ID NO: 2):

| | Location in the | Location in |
|---|---|---|
| Location in GRCh38/hg38 | causative gene | the regen |
| human reference genome | (SEQ ID NO: 2) | (SEQ ID NO: 34) |
| chr11: 17444455 | 32391 | 67 |
| chr11: 17444440 | 32406 | 82 |
| chr11: 17444436 | 32410 | 86 |

The branch point that can be involved in pseudo-exon-
type aberrant splicing caused by the mutation (chr5:
112790640T > C) in the APC gene (SEQ ID NO: 3):

| | Location in the | Location in |
|---|---|---|
| Location in GRCh38/hg38 | causative gene | the regen |
| human reference genome | (SEQ ID NO: 3) | (SEQ ID NO: 35) |
| chr5: 112790443 | 82946 | 51 |
| chr5: 112790465 | 82968 | 73 |
| chr5: 112790478 | 82981 | 86 |
| chr5: 112790493 | 82996 | 101 |

The branch point that can be involved in pseudo-exon-
type aberrant splicing caused by the mutation (chr5:
112822720A > G, chr5: 112822722C > T, chr5: 112822726A >
T, or chr5: 112822734G > A) in the APC gene (SEQ ID NO: 3):

| | Location in the | Location in |
|---|---|---|
| Location in GRCh38/hg38 | causative gene | the regen |
| human reference genome | (SEQ ID NO: 3) | (SEQ ID NO: 36) |
| chr5: 112822544 | 115047 | 27 |
| chr5: 112822547 | 115050 | 30 |
| chr5: 112822555 | 115058 | 38 |
| chr5: 112822569 | 115072 | 52 |
| chr5: 112822577 | 115080 | 60 |
| chr5: 112822583 | 115086 | 66 |
| chr5: 112822588 | 115091 | 71 |
| chr5: 112822611 | 115114 | 94 |
| chr5: 112822616 | 115119 | 99 |

The branch point that can be involved in pseudo-exon-
type aberrant splicing caused by the mutation (chr5:
112779849G > A) in the APC gene (SEQ ID NO: 3):

| | Location in the | Location in |
|---|---|---|
| Location in GRCh38/hg38 | causative gene | the regen |
| human reference genome | (SEQ ID NO: 3) | (SEQ ID NO: 37) |
| chr5: 112779603 | 72106 | 40 |
| chr5: 112779606 | 72109 | 43 |
| chr5: 112779614 | 72117 | 51 |
| chr5: 112779618 | 72121 | 55 |
| chr5: 112779626 | 72129 | 63 |
| chr5: 112779633 | 72136 | 70 |
| chr5: 112779642 | 72145 | 79 |
| chr5: 112779660 | 72163 | 97 |
| chr5: 112779666 | 72169 | 103 |

The branch point that can be involved in pseudo-exon-
type aberrant splicing caused by the mutation (chr11:
108270483_108270486del) in the ATM gene (SEQ ID NO: 4):

| | Location in the | Location in |
|---|---|---|
| Location in GRCh38/hg38 | causative gene | the regen |
| human reference genome | (SEQ ID NO: 4) | (SEQ ID NO: 38) |
| chr11: 108270378 | 47312 | 28 |
| chr11: 108270386 | 47320 | 36 |
| chr11: 108270387 | 47321 | 37 |
| chr11: 108270402 | 47336 | 52 |
| chr11: 108270405 | 47339 | 55 |
| chr11: 108270408 | 47342 | 58 |
| chr11: 108270411 | 47345 | 61 |
| chr11: 108270415 | 47349 | 65 |
| chr11: 108270419 | 47353 | 69 |
| chr11: 108270445 | 47379 | 95 |
| chr11: 108270451 | 47385 | 101 |

The branch point that can be involved in pseudo-exon-
type aberrant splicing caused by the mutation (chr11:
108309110A > G) in the ATM gene (SEQ ID NO: 4):

| | Location in the | Location in |
|---|---|---|
| Location in GRCh38/hg38 | causative gene | the regen |
| human reference genome | (SEQ ID NO: 4) | (SEQ ID NO: 39) |
| chr11: 108308880 | 85814 | 32 |
| chr11: 108308892 | 85826 | 44 |
| chr11: 108308901 | 85835 | 53 |
| chr11: 108308902 | 85836 | 54 |
| chr11: 108308947 | 85881 | 99 |
| chr11: 108308954 | 85888 | 106 |

The branch point that can be involved in pseudo-exon-
type aberrant splicing caused by the mutation (chr17:
43086839G > A) in the BRCA1 gene (SEQ ID NO: 5):

| | Location in the | Location in |
|---|---|---|
| Location in GRCh38/hg38 | causative gene | the regen |
| human reference genome | (SEQ ID NO: 5) | (SEQ ID NO: 40) |
| chr17: 43087041 | 38324 | 34 |
| chr17: 43087029 | 38336 | 46 |
| chr17: 43087028 | 38337 | 47 |
| chr17: 43087021 | 38344 | 54 |
| chr17: 43087004 | 88361 | 71 |
| chr17: 43086992 | 38373 | 83 |
| chr17: 43086981 | 38384 | 94 |

The branch point that can be involved in pseudo-exon-
type aberrant splicing caused by the mutation (chr13:
32345247T > G) in the BRCA2 gene (SEQ ID NO: 6):

| | Location in the | Location in |
|---|---|---|
| Location in GRCh38/hg38 | causative gene | the regen |
| human reference genome | (SEQ ID NO: 6) | (SEQ ID NO: 41) |
| chr13: 32345085 | 29578 | 58 |
| chr13: 32345091 | 29584 | 64 |
| chr13: 32345096 | 29589 | 69 |
| chr13: 32345104 | 29597 | 77 |
| chr13: 32345122 | 29615 | 95 |
| chr13: 32345131 | 29624 | 104 |

The branch point that can be involved in pseudo-exon-
type aberrant splicing caused by the mutation (chr17:
61781503T > A) in the BRIP1 gene (SEQ ID NO: 7):

| | Location in the | Location in |
|---|---|---|
| Location in GRCh38/hg38 | causative gene | the regen |
| human reference genome | (SEQ ID NO: 7) | (SEQ ID NO: 42) |
| chr17: 61782558 | 80971 | 30 |
| chr17: 61782554 | 80975 | 34 |
| chr17: 61782541 | 80988 | 47 |
| chr17: 61782524 | 81005 | 64 |
| chr17: 61782523 | 81006 | 65 |
| chr17: 61782514 | 81015 | 74 |
| chr17: 61782503 | 81026 | 85 |

The branch point that can be involved in pseudo-exon-
type aberrant splicing caused by the mutation (chr12:
88101183T > C) in the CEP290 gene (SEQ ID NO: 8):

| | Location in the | Location in |
|---|---|---|
| Location in GRCh38/hg38 | causative gene | the regen |
| human reference genome | (SEQ ID NO: 8) | (SEQ ID NO: 43) |
| chr12: 88101399 | 40690 | 37 |
| chr12: 88101381 | 40708 | 55 |
| chr12: 88101372 | 40717 | 64 |
| chr12: 88101343 | 40746 | 93 |
| chr12: 88101332 | 40757 | 104 |

The branch point that can be involved in pseudo-exon-
type aberrant splicing caused by the mutation (chr7:
117578327A > G) in the CFTR gene (SEQ ID NO: 9):

| | Location in the | Location in |
|---|---|---|
| Location in GRCh38/hg38 | causative gene | the regen |
| human reference genome | (SEQ ID NO: 9) | (SEQ ID NO: 44) |
| chr7: 117578130 | 98106 | 32 |
| chr7: 117578142 | 98118 | 44 |
| chr7: 117578149 | 98125 | 51 |
| chr7: 117578164 | 98140 | 66 |
| chr7: 117578182 | 98158 | 84 |

The branch point that can be involved in pseudo-exon-
type aberrant splicing caused by the mutation (chr7:
117589467A > G) in the CFTR gene (SEQ ID NO: 9):

| | Location in the | Location in |
|---|---|---|
| Location in GRCh38/hg38 | causative gene | the regen |
| human reference genome | (SEQ ID NO: 9) | (SEQ ID NO: 45) |
| chr7: 117589329 | 109305 | 32 |
| chr7: 117589330 | 109306 | 33 |
| chr7: 117589350 | 109326 | 53 |
| chr7: 117589351 | 109327 | 54 |
| chr7: 117589362 | 109338 | 65 |
| chr7: 117589363 | 109339 | 66 |
| chr7: 117589373 | 109349 | 76 |
| chr7: 117589374 | 109350 | 77 |
| chr7: 117589384 | 109360 | 87 |
| chr7: 117589385 | 109361 | 88 |
| chr7: 117589396 | 109372 | 99 |

The branch point that can be involved in pseudo-exon-
type aberrant splicing caused by the mutation (chr7:
117639961C > T) in the CFTR gene (SEQ ID NO: 9):

| | Location in the | Location in |
|---|---|---|
| Location in GRCh38/hg38 | causative gene | the regen |
| human reference genome | (SEQ ID NO: 9) | (SEQ ID NO: 46) |
| chr7: 117639791 | 159767 | 36 |
| chr7: 117639804 | 159780 | 49 |
| chr7: 117639807 | 159783 | 52 |
| chr7: 117639808 | 159784 | 53 |
| chr7: 117639811 | 159787 | 56 |
| chr7: 117639814 | 159790 | 59 |
| chr7: 117639815 | 159791 | 60 |
| chr7: 117639828 | 159804 | 73 |
| chr7: 117639836 | 159812 | 81 |
| chr7: 117639844 | 159820 | 89 |
| chr7: 117639848 | 159824 | 93 |
| chr7: 117639851 | 159827 | 96 |
| chr7: 117639854 | 159830 | 99 |

The branch point that can be involved in pseudo-exon-
type aberrant splicing caused by the mutation (chr3:
150942410A > C) in the CLRN1 gene (SEQ ID NO: 10):

| | Location in the | Location in |
|---|---|---|
| Location in GRCh38/hg38 | causative gene | the regen |
| human reference genome | (SEQ ID NO: 10) | (SEQ ID NO: 47) |
| chr3: 150942716 | 30284 | 44 |
| chr3: 150942694 | 30306 | 66 |
| chr3: 150942687 | 30313 | 73 |
| chr3: 150942681 | 30319 | 79 |
| chr3: 150942661 | 30339 | 99 |

The branch point that can be involved in pseudo-exon-
type aberrant splicing caused by the mutation (chr8:
86605416C > T) in the CNGB3 gene (SEQ ID NO: 11):

| | Location in the | Location in |
|---|---|---|
| Location in GRCh38/hg38 | causative gene | the regen |
| human reference genome | (SEQ ID NO: 11) | (SEQ ID NO: 48) |
| chr8: 86605542 | 138093 | 32 |
| chr8: 86605541 | 138094 | 33 |
| chr8: 86605538 | 138097 | 36 |
| chr8: 86605528 | 138107 | 46 |
| chr8: 86605525 | 138110 | 49 |
| chr8: 86606524 | 138111 | 50 |
| chr8: 86605520 | 138115 | 54 |
| chr8: 86605515 | 138120 | 59 |
| chr8: 86605499 | 138136 | 75 |
| chr8: 86605490 | 138145 | 84 |
| chr8: 86605486 | 138149 | 88 |
| chr8: 86605481 | 138154 | 93 |
| chr8: 86605473 | 138162 | 101 |
| chr8: 86605472 | 138163 | 102 |

The branch point that can be involved in pseudo-exon-
type aberrant splicing caused by the mutation (chr21:
45989967C > T) in the COL6A1 gene (SEQ ID NO: 12):

| | Location in the | Location in |
|---|---|---|
| Location in GRCh38/hg38 | causative gene | the regen |
| human reference genome | (SEQ ID NO: 12) | (SEQ ID NO: 49) |
| chr21: 45989856 | 8087 | 83 |
| chr21: 45989871 | 8102 | 98 |

The branch point that can be involved in pseudo-exon-
type aberrant splicing caused by the mutation (chr2:
71661900G > T) in the DYSF gene (SEQ ID NO: 13):

| | Location in the | Location in |
|---|---|---|
| Location in GRCh38/hg38 | causative gene | the regen |
| human reference genome | (SEQ ID NO: 13) | (SEQ ID NO: 50) |
| chr2: 71661637 | 208077 | 36 |
| chr2: 71661655 | 208095 | 54 |
| chr2: 71661664 | 208104 | 63 |

The branch point that can be involved in pseudo-exon-
type aberrant splicing caused by the mutation (chr9:
105606576G > T) in the FKTN gene (SEQ ID NO: 14):

| | Location in the | Location in |
|---|---|---|
| Location in GRCh38/hg38 | causative gene | the regen |
| human reference genome | (SEQ ID NO: 14) | (SEQ ID NO: 51) |
| chr9: 105606413 | 48284 | 27 |
| chr9: 105606414 | 48285 | 28 |
| chr9: 105606420 | 48291 | 34 |
| chr9: 105606428 | 48299 | 42 |
| chr9: 105606429 | 48300 | 43 |
| chr9: 105606441 | 48312 | 55 |
| chr9: 105606446 | 48317 | 60 |
| chr9: 105606460 | 48331 | 74 |
| chr9: 105606469 | 48340 | 83 |
| chr9: 105606488 | 48359 | 102 |

The branch point that can be involved in pseudo-exon-
type aberrant splicing caused by the mutation (chr17:
80104542T > G) in the GAA gene (SEQ ID NO: 15):

| | Location in the | Location in |
|---|---|---|
| Location in GRCh38/hg38 | causative gene | the regen |
| human reference genome | (SEQ ID NO: 15) | (SEQ ID NO: 52) |
| chr17: 80104315 | 2735 | 35 |
| chr17: 80104322 | 2742 | 42 |
| chr17: 80104329 | 2749 | 49 |

The branch point that can be involved in pseudo-exon-
type aberrant splicing caused by the mutation (chr9:
34649954A > G) in the GALT gene (SEQ ID NO: 16):

| | Location in the | Location in |
|---|---|---|
| Location in GRCh38/hg38 | causative gene | the regen |
| human reference genome | (SEQ ID NO: 16) | (SEQ ID NO: 53) |
| chr9: 34649864 | 3190 | 30 |
| chr9: 34649874 | 3200 | 40 |
| chr9: 34649877 | 3203 | 43 |
| chr9: 34649886 | 3212 | 52 |
| chr9: 34649926 | 3252 | 92 |
| chr9: 34649933 | 3259 | 99 |
| chr9: 34649939 | 3265 | 105 |

The branch point that can be involved in pseudo-exon-
type aberrant splicing caused by the mutation (chr5:
42700794A > G) in the GHR gene (SEQ ID NO: 17):

| | Location in the | Location in |
|---|---|---|
| Location in GRCh38/hg38 | causative gene | the regen |
| human reference genome | (SEQ ID NO: 17) | (SEQ ID NO: 54) |
| chr5: 42700599 | 277161 | 33 |
| chr5: 42700602 | 277164 | 36 |
| chr5: 42700608 | 277170 | 42 |
| chr5: 42700620 | 277182 | 54 |
| chr5: 42700643 | 277205 | 77 |
| chr5: 42700660 | 277222 | 94 |
| chr5: 42700664 | 277226 | 98 |
| chr5: 42700670 | 277232 | 104 |

The branch point that can be involved in pseudo-exon-
type aberrant splicing caused by the mutation (chrX:
101399747C > T) in the GLA gene (SEQ ID NO: 18):

| | Location in the | Location in |
|---|---|---|
| Location in GRCh38/hg38 | causative gene | the regen |
| human reference genome | (SEQ ID NO: 18) | (SEQ ID NO: 55) |
| chrX: 101399867 | 8059 | 54 |
| chrX: 101399859 | 8067 | 62 |
| chrX: 101399858 | 8068 | 63 |
| chrX: 101399838 | 8088 | 83 |
| chrX: 101399831 | 8095 | 90 |
| chrX: 101399828 | 8098 | 93 |
| chrX: 101399823 | 8103 | 98 |
| chrX: 101399822 | 8104 | 99 |
| chrX: 101399814 | 8112 | 107 |

The branch point that can be involved in pseudo-exon-
type aberrant splicing caused by the mutation (chr4:
108023948A > G) in the HADH gene (SEQ ID NO: 19):

| | Location in the | Location in |
|---|---|---|
| Location in GRCh38/hg38 | causative gene | the regen |
| human reference genome | (SEQ ID NO: 19) | (SEQ ID NO: 56) |
| chr4: 108023860 | 33972 | 32 |
| chr4: 108023863 | 33975 | 35 |
| chr4: 108023878 | 33990 | 50 |
| chr4: 108023881 | 33993 | 53 |
| chr4: 108023885 | 33997 | 57 |
| chr4: 108023890 | 34002 | 62 |
| chr4: 108023905 | 34017 | 77 |
| chr4: 108023921 | 34033 | 93 |
| chr4: 108023931 | 34043 | 103 |

The branch point that can be involved in pseudo-exon-
type aberrant splicing caused by the mutation (chr11:
5225872A > C, chr11: 5225832G > C, or chr11:
5225923G > A) in the HBB gene (SEQ ID NO: 20):

| | Location in the | Location in |
|---|---|---|
| Location in GRCh38/hg38 | causative gene | the regen |
| human reference genome | (SEQ ID NO: 20) | (SEQ ID NO: 57) |
| chr11: 5226078 | 994 | 40 |
| chr11: 5226073 | 999 | 45 |
| chr11: 5226072 | 1000 | 46 |
| chr11: 5226053 | 1019 | 65 |
| chr11: 5226052 | 1020 | 66 |
| chr11: 5226036 | 1036 | 82 |
| chr11: 5226031 | 1041 | 87 |
| chr11: 5226024 | 1048 | 94 |
| chr11: 5226012 | 1060 | 106 |

The branch point that can be involved in pseudo-exon-
type aberrant splicing caused by the mutation (chr19:
11120625G > T) in the LDLR gene (SEQ ID NO: 21):

| | Location in the | Location in |
|---|---|---|
| Location in GRCh38/hg38 | causative gene | the regen |
| human reference genome | (SEQ ID NO: 21) | (SEQ ID NO: 58) |
| chr19: 11120329 | 30867 | 80 |
| chr19: 11120332 | 30870 | 83 |
| chr19: 11120349 | 30887 | 100 |

The branch point that can be involved in pseudo-exon-
type aberrant splicing caused by the mutation (chr19:
11122956G > A) in the LDLR gene (SEQ ID NO: 21):

| | Location in the | Location in |
|---|---|---|
| Location in GRCh38/hg38 | causative gene | the regen |
| human reference genome | (SEQ ID NO: 21) | (SEQ ID NO: 59) |
| chr19: 11122732 | 33270 | 31 |
| chr19: 11122742 | 33280 | 41 |
| chr19: 11122761 | 33299 | 60 |
| chr19: 11122775 | 33313 | 74 |
| chr19: 11122776 | 33314 | 75 |
| chr19: 11122784 | 33322 | 83 |
| chr19: 11122787 | 33325 | 86 |
| chr19: 11122788 | 33326 | 87 |
| chr19: 11122791 | 33329 | 90 |
| chr19: 11122794 | 33332 | 93 |

The branch point that can be involved in pseudo-exon-
type aberrant splicing caused by the mutation (chr11:
47343314C > T) in the MYBPC3 gene (SEQ ID NO: 22):

| | Location in the | Location in |
|---|---|---|
| Location in GRCh38/hg38 | causative gene | the regen |
| human reference genome | (SEQ ID NO: 22) | (SEQ ID NO: 60) |
| chr11: 47343403 | 9300 | 30 |
| chr11: 47343387 | 9316 | 46 |
| chr11: 47343366 | 9337 | 67 |

The branch point that can be involved in pseudo-exon-
type aberrant splicing caused by the mutation (chr6:
51882440T > C) in the PKHD1 gene (SEQ ID NO: 23):

| | Location in the | Location in |
|---|---|---|
| Location in GRCh38/hg38 | causative gene | the regen |
| human reference genome | (SEQ ID NO: 23) | (SEQ ID NO: 61) |
| chr6: 51882633 | 204983 | 44 |
| chr6: 51882602 | 205014 | 75 |
| chr6: 51882595 | 205021 | 82 |
| chr6: 51882586 | 205030 | 91 |

The branch point that can be involved in pseudo-exon-
type aberrant splicing caused by the mutation (chr14:
21321131T > G) in the RPGRIP1 gene (SEQ ID NO: 24):

| | Location in the | Location in |
|---|---|---|
| Location in GRCh38/hg38 | causative gene | the regen |
| human reference genome | (SEQ ID NO: 24) | (SEQ ID NO: 62) |
| chr14: 21320926 | 40844 | 38 |
| chr14: 21320950 | 40868 | 62 |
| chr14: 21320953 | 40871 | 65 |
| chr14: 21320986 | 40904 | 98 |
| chr14: 21320989 | 40907 | 101 |

The branch point that can be involved in pseudo-exon-
type aberrant splicing caused by the mutation (chr16:
56883858C > T) in the SLC12A3 gene (SEQ ID NO: 25):

| | Location in the | Location in |
|---|---|---|
| Location in GRCh38/hg38 | causative gene | the regen |
| human reference genome | (SEQ ID NO: 25) | (SEQ ID NO: 63) |
| chr16: 56883532 | 18326 | 34 |
| chr16: 56883554 | 18348 | 56 |
| chr16: 56883571 | 18365 | 73 |
| chr16: 56883587 | 18381 | 89 |

The branch point that can be involved in pseudo-exon-
type aberrant splicing caused by the mutation (chr16:
56893307C > T) in the SLC12A3 gene (SEQ ID NO: 25):

| | Location in the | Location in |
|---|---|---|
| Location in GRCh38/hg38 | causative gene | the regen |
| human reference genome | (SEQ ID NO: 25) | (SEQ ID NO: 64) |
| chr16: 56893142 | 27936 | 47 |
| chr16: 56893174 | 27968 | 79 |
| chr16: 56893179 | 27973 | 84 |

The branch point that can be involved in pseudo-exon-
type aberrant splicing caused by the mutation (chr1:
215891198T > C) in the USH2A gene (SEQ ID NO: 26):

| | Location in the | Location in |
|---|---|---|
| Location in GRCh38/hg38 | causative gene | the regen |
| human reference genome | (SEQ ID NO: 26) | (SEQ ID NO: 65) |
| chr1: 215891437 | 532012 | 34 |
| chr1: 215891434 | 532015 | 37 |
| chr1: 215891429 | 532020 | 42 |
| chr1: 215891428 | 532021 | 43 |
| chr1: 215891420 | 532029 | 51 |
| chr1: 215891415 | 532034 | 56 |
| chr1: 215891398 | 532051 | 73 |
| chr1: 215891391 | 532058 | 80 |
| chr1: 215891390 | 532059 | 81 |
| chr1: 215891366 | 532083 | 105 |

The branch point that can be involved in pseudo-exon-
type aberrant splicing caused by the mutation (chr1:
215794441T > C) in the USH2A gene (SEQ ID NO: 26):

| | Location in the | Location in |
|---|---|---|
| Location in GRCh38/hg38 | causative gene | the regen |
| human reference genome | (SEQ ID NO: 26) | (SEQ ID NO: 66) |
| chr1: 215794677 | 628772 | 40 |
| chr1: 215794674 | 628775 | 43 |
| chr1: 215794671 | 628778 | 46 |
| chr1: 215794660 | 628789 | 57 |
| chr1: 215794659 | 628790 | 58 |
| chr1: 215794652 | 628797 | 65 |
| chr1: 215794644 | 628805 | 73 |
| chr1: 215794620 | 628829 | 97 |
| chr1: 215794611 | 628838 | 106 |

The branch point that can be involved in pseudo-exon-
type aberrant splicing caused by the mutation (chr8:
31108591A > G) in the WRN gene (SEQ ID NO: 27):

| | Location in the | Location in |
|---|---|---|
| Location in GRCh38/hg38 | causative gene | the regen |
| human reference genome | (SEQ ID NO: 27) | (SEQ ID NO: 67) |
| chr8: 31108392 | 74583 | 28 |
| chr8: 31108395 | 74586 | 31 |
| chr8: 31108410 | 74601 | 46 |
| chr8: 31108413 | 74604 | 49 |
| chr8: 31108431 | 74622 | 67 |
| chr8: 31108438 | 74629 | 74 |
| chr8: 31108450 | 74641 | 86 |
| chr8: 31108453 | 74644 | 89 |
| chr8: 31108456 | 74647 | 92 |
| chr8: 31108457 | 74648 | 93 |
| chr8: 31108463 | 74654 | 99 |
| chr8: 31108467 | 74658 | 103 |

In one or more embodiments, the branch point that can be involved in pseudo-exon-type aberrant splicing caused by the mutation (chr1:94084225G>A) in the ABCA4 gene may be an adenine base at chr1:94084303.

In one or more embodiments, the branch point that can be involved in pseudo-exon-type aberrant splicing caused by the mutation (chr1:94081224C>G) in the ABCA4 gene may be an adenine base at chr1:94081251.

In one or more embodiments, the branch point that can be involved in pseudo-exon-type aberrant splicing caused by the mutation (chr1:94062142G>C) in the ABCA4 gene may be an adenine base at chr1:94062224.

In one or more embodiments, the branch point that can be involved in pseudo-exon-type aberrant splicing caused by the mutation (chr1:94028345T>C) in the ABCA4 gene may be an adenine base at chr1:94028466.

In one or more embodiments, the branch point that can be involved in pseudo-exon-type aberrant splicing caused by the mutation (chr1:94028345T>C) in the ABCA4 gene may be an adenine base at chr1:94028506.

In one or more embodiments, the branch point that can be involved in pseudo-exon-type aberrant splicing caused by the mutation (chr1:94027444C>T) in the ABCA4 gene may be an adenine base at chr1:94027606.

In one or more embodiments, the branch point that can be involved in pseudo-exon-type aberrant splicing caused by a mutation (chr11:17444325T>C) in the ABCC8 gene may be an adenine base at chr11:17444455.

In one or more embodiments, the branch point that can be involved in pseudo-exon-type aberrant splicing caused by the mutation (chr5:112790640T>G) in the APC gene may be an adenine base at chr5:112790493.

In one or more embodiments, the branch point that can be involved in pseudo-exon-type aberrant splicing caused by the mutation (chr5:112822720A>G, chr5:112822722C>T, chr5:112822726A>T, or chr5:112822734G>A) in the APC gene may be an adenine base at chr5:112822616.

In one or more embodiments, the branch point that can be involved in pseudo-exon-type aberrant splicing caused by the mutation (chr5:112779849G>A) in the APC gene may be an adenine base at chr5:112779660.

In one or more embodiments, the branch point that can be involved in pseudo-exon-type aberrant splicing caused by the mutation (chr11:108270483_108270486del) in the ATM gene may be an adenine base at chr11:108270408.

In one or more embodiments, the branch point that can be involved in pseudo-exon-type aberrant splicing caused by the mutation (chr11:108309110A>G) in the ATM gene may be an adenine base at chr11:108308954.

In one or more embodiments, the branch point that can be involved in pseudo-exon-type aberrant splicing caused by the mutation (chr17:43086839G>A) in the BRCA1 gene may be an adenine base at chr17:43086981.

In one or more embodiments, the branch point that can be involved in pseudo-exon-type aberrant splicing caused by the mutation (chr13:32345247T>G) in the BRCA2 gene may be an adenine base at chr13:32345085.

In one or more embodiments, the branch point that can be involved in pseudo-exon-type aberrant splicing caused by the mutation (chr17:61781503T>A) in the BRIP1 gene may be an adenine base at chr17:61782523.

In one or more embodiments, the branch point that can be involved in pseudo-exon-type aberrant splicing caused by the mutation (chr12:88101183T>C) in the CEP290 gene may be an adenine base at chr12:88101332.

In one or more embodiments, the branch point that can be involved in pseudo-exon-type aberrant splicing caused by the mutation (chr7:117578327A>G) in the CFTR gene may be an adenine base at chr7:117578182.

In one or more embodiments, the branch point that can be involved in pseudo-exon-type aberrant splicing caused by the mutation (chr7:117589467A>G) in the CFTR gene may be an adenine base at chr7:117589396.

In one or more embodiments, the branch point that can be involved in pseudo-exon-type aberrant splicing caused by the mutation (chr7:117639961C>T) in the CFTR gene may be an adenine base at chr7:117639851.

In one or more embodiments, the branch point that can be involved in pseudo-exon-type aberrant splicing caused by the mutation (chr3:150942410A>C) in the CLRN1 gene may be an adenine base at chr3:150942661.

In one or more embodiments, the branch point that can be involved in pseudo-exon-type aberrant splicing caused by the mutation (chr8:86605416C>T) in the CNGB3 gene may be an adenine base at chr8:86605515.

In one or more embodiments, the branch point that can be involved in pseudo-exon-type aberrant splicing caused by the mutation (chr21:45989967C>T) in the COL6A1 gene may be an adenine base at chr21:45989871.

In one or more embodiments, the branch point that can be involved in pseudo-exon-type aberrant splicing caused by the mutation (chr2:71661900G>T) in the DYSF gene may be an adenine base at chr2:71661637.

In one or more embodiments, the branch point that can be involved in pseudo-exon-type aberrant splicing caused by the mutation (chr9:105606576G>T) in the FKTN gene may be an adenine base at chr9:105606469.

In one or more embodiments, the branch point that can be involved in pseudo-exon-type aberrant splicing caused by the mutation (chr17:80104542T>G) in the GAA gene may be an adenine base at chr17:80104322.

In one or more embodiments, the branch point that can be involved in pseudo-exon-type aberrant splicing caused by the mutation (chr9:34649954A>G) in the GALT gene may be an adenine base at chr9:34649926.

In one or more embodiments, the branch point that can be involved in pseudo-exon-type aberrant splicing caused by the mutation (chr5:42700794A>G) in the GHR gene may be an adenine base at chr5:42700670.

In one or more embodiments, the branch point that can be involved in pseudo-exon-type aberrant splicing caused by the mutation (chrX:101399747C>T) in the GLA gene may be an adenine base at chrX: 101399822.

In one or more embodiments, the branch point that can be involved in pseudo-exon-type aberrant splicing caused by the mutation (chr4:108023948A>G) in the HADH gene may be an adenine base at chr4:108023921.

In one or more embodiments, the branch point that can be involved in pseudo-exon-type aberrant splicing caused by the mutation (chr11:5225872A>C, chr11:5225832G>C, or chr11:5225923G>A) in the EBB gene may be an adenine base at chr11:5226012.

In one or more embodiments, the branch point that can be involved in pseudo-exon-type aberrant splicing caused by the mutation (chr19:11120625G>T) in the LDLR gene may be an adenine base at chr19:11120349.

In one or more embodiments, the branch point that can be involved in pseudo-exon-type aberrant splicing caused by the mutation (chr19:11122956G>A) in the LDLR gene may be an adenine base at chr19:11122761.

In one or more embodiments, the branch point that can be involved in pseudo-exon-type aberrant splicing caused by the mutation (chr11:47343314C>T) in the MYBPC3 gene may be an adenine base at chr11:47343366.

In one or more embodiments, the branch point that can be involved in pseudo-exon-type aberrant splicing caused by the mutation (chr6:51882440T>C) in the PKHD1 gene may be an adenine base at chr6:51882586.

In one or more embodiments, the branch point that can be involved in pseudo-exon-type aberrant splicing caused by the mutation (chr14:21321131T>G) in the RPGRIP1 gene may be an adenine base at chr14:21320953.

In one or more embodiments, the branch point that can be involved in pseudo-exon-type aberrant splicing caused by the mutation (chr16:56883858C>T) in the SLC12A3 gene may be an adenine base at chr16:56883587.

In one or more embodiments, the branch point that can be involved in pseudo-exon-type aberrant splicing caused by the mutation (chr16:56893307C>T) in the SLC12A3 gene may be an adenine base at chr16:56893174.

In one or more embodiments, the branch point that can be involved in pseudo-exon-type aberrant splicing caused by the mutation (chr1:215891198T>C) in the USH2A gene may be an adenine base at chr1:215891390.

In one or more embodiments, the branch point that can be involved in pseudo-exon-type aberrant splicing caused by the mutation (chr1:215794441T>C) in the USH2A gene may be an adenine base at chr1:215794611.

In one or more embodiments, the branch point that can be involved in pseudo-exon-type aberrant splicing caused by the mutation in the WRN gene (chr8:31108591A>G) may be an adenine base at chr8:31108457.

In one or more embodiments, the target sequence of the antisense oligonucleotide of the present disclosure is a sequence including a branch point in pseudo-exon-type aberrant splicing. The “target sequence” in the present disclosure refers to a target nucleic acid sequence to which the antisense oligonucleotide of the present disclosure hybridizes, and can also be referred to as a sequence with which the antisense oligonucleotide forms a complementary hybrid. The target sequence in the present disclosure is a pre-mRNA sequence of each gene in one or more embodiments. The “pre-mRNA” in the present disclosure can also be referred to as an mRNA precursor, and refers to an RNA that includes both exons and introns.

Although the antisense oligonucleotide of the present disclosure includes a sequence complementary to the target sequence in one or more embodiments, such a sequence need not necessarily be perfectly complementary to the target sequence. In one or more embodiments, the antisense oligonucleotide of the present disclosure may include mismatches to the extent that the antisense oligonucleotide can form a hybrid with a target sequence including a branch point in pseudo-exon-type aberrant splicing. In one or more embodiments, the antisense oligonucleotide of the present disclosure need only be such that at least 40% of bases thereof are complementary to the target sequence, and preferably at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of bases thereof are complementary to the target sequence.

In one or more embodiments, examples of the target sequence in the present disclosure include a base sequence consisting of 10 to 50 successive bases in any of regions (1) to (40) (base sequences represented by SEQ ID NOs: 28 to 67) to be described below and including a branch point in pseudo-exon-type aberrant splicing. In one or more embodiments, the branch point is as described above.

In one or more embodiments, the length of the target sequence can be determined according to the length and the like of the antisense oligonucleotide intended to be obtained. In one or more embodiments, the length of the target sequence is 10 to 50 bases. In one or more embodiments, the length of the target sequence is 11 bases or more, 12 bases or more, 13 bases or more, 14 bases or more, or 15 bases or more. In one or more embodiments, the length of the target sequence is 45 bases or less, 40 bases or less, 39 bases or less, 38 bases or less, 37 bases or less, 36 bases or less, 35 bases or less, 34 bases or less, 33 bases or less, 32 bases or less, 30 bases or less, 29 bases or less, 28 bases or less, 27 bases or less, 26 bases or less, or 25 bases or less.

In one or more embodiments, the antisense oligonucleotide of the present disclosure is an antisense oligonucleotide whose target sequence is a sequence including at least one branch point selected from the group consisting of the above-described branch points (adenine bases) that can be involved in pseudo-exon-type aberrant splicing. Since the target sequence of the antisense oligonucleotide of the present disclosure is a sequence including a branch point(s) (adenine bases) that can be involved in pseudo-exon-type aberrant splicing in one or more embodiments, the antisense oligonucleotide of the present disclosure has activity capable of suppressing and/or correcting pseudo-exon-type splicing defects in which the branch point(s) is involved.

In one or more embodiments, the antisense oligonucleotide of the present disclosure is an RNA molecule that is complementary to a target sequence consisting of 10 to 50 successive bases in the base sequence represented by any of SEQ ID NOs: 28 to 67 (any of regions (1) to (40)) and is designed so as to bind to a sequence including a branch point(s) in pseudo-exon-type aberrant splicing present in the target sequence, thereby correcting the pseudo-exon-type aberrant splicing. In one or more embodiments, examples of the branch point include those described above.

SEQ ID NO: 28 is an example of a region where a branch point that can be involved in pseudo-exon-type aberrant splicing is present in the ABCA4 gene, and represents the base sequence of a region (1) from 94084339th to 94084229th bases (36810th to 36920th bases in SEQ ID NO: 1) in the base sequence of human chromosome 1 in the GRCh38/hg38 human reference genome list.

(SEQ ID NO: 28)
AAGTCCAGCTGGTTAAAAGGCACATGCCCAGTGCTCACTTCACACCT
ACTCAGGAAGCACACTTGAGTTGGAAAACCACTGTCTTTACACTTAG
AACTCAGTCCTACATGA

SEQ ID NO: 29 is another example of a region where a branch point that can be involved in pseudo-exon-type aberrant splicing is present in the ABCA4 gene, and represents the base sequence of a region (2) from 94081341st to 94081231st bases (39808th to 39918th bases in SEQ ID NO: 1) in the base sequence of human chromosome 1 in the GRCh38/hg38 human reference genome list.

(SEQ ID NO: 29)
ATTTGCCCAAGGACACATTCCCAACGAATTCAAATAAAGGAGACTAG
AAGAAGAGAGGCTATACTACAGTGCTCTAGGGGTCACTCTGTGATTT
GTTGTTGTTGTTGTTGT

SEQ ID NO: 30 is still another example of a region where a branch point that can be involved in pseudo-exon-type aberrant splicing is present in the ABCA4 gene, and represents the base sequence of a region (3) from 94062301st to 94062191st bases (58848th to 58958th bases in SEQ ID NO: 1) in the base sequence of human chromosome 1 in the GRCh38/hg38 human reference genome list.

(SEQ ID NO: 30)
TGGGGGAGGGGACAAATTCCCCACTATGTAGTATGTTTGGTATGTGG
AAGGGTTCTGGTCAGAATGTTTGCCCAATGATTGCCACATCAGCATT
CATTTTGGACTCTGTAT

SEQ ID NO: 31 is still another example of a region where a branch point that can be involved in pseudo-exon-type aberrant splicing is present in the ABCA4 gene, and represents the base sequence of a region (4) from 94028532nd to 94028422nd bases (92617th to 92727th bases in SEQ ID NO: 1) in the base sequence of human chromosome 1 in the GRCh38/hg38 human reference genome list.

(SEQ ID NO: 31)
ATGGAAATGTGTTTACACACTTATTAACAGTCTTAATTAAGAAGCTC
TCCATGTGCTGTGTCTCTAACATCTGCAGGTATGTACACAAATACAT
GCACAGCCAGCATCCAT

SEQ ID NO: 32 is still another example of a region where a branch point that can be involved in pseudo-exon-type aberrant splicing is present in the ABCA4 gene, and represents the base sequence of a region (5) from 94028576th to 94028466th bases (92573rd to 92683rd bases in SEQ ID NO: 1) in the base sequence of human chromosome 1 in the GRCh38/hg38 human reference genome list.

(SEQ ID NO: 32)
TTTTAGTTTTCACCATTATAAGCAATGCTATGATGTACATTCAAATG
GAAATGTGTTTACACACTTATTAACAGTCTTAATTAAGAAGCTCTCC
ATGTGCTGTGTCTCTAA

SEQ ID NO: 33 is an example of a region where a branch point that can be involved in pseudo-exon-type aberrant splicing is present in the ABCA4 gene, and represents the base sequence of a region (6) from 94027674th to 94027564th bases (93475th to 93585th bases in SEQ ID NO: 1) in the base sequence of human chromosome 1 in the GRCh38/hg38 human reference genome list.

(SEQ ID NO: 33)
TATCACAGTCTGGTTTATAAATGGTTCTAGGCCAAGAACACCCGATC
CCTGCTCTTTTTTATATTCTAAAGCATGTATCTTTATATTTCTCAAG
CAATATTTTCTCTCTTT

SEQ ID NO: 34 is an example of a region where a branch point that can be involved in pseudo-exon-type aberrant splicing is present in the ABCC8 gene, and represents the base sequence of a region (7) from 17444521st to 17444411th bases (32325th to 32435th bases in SEQ ID NO: 2) in the base sequence of human chromosome 11 in the GRCh38/hg38 human reference genome list.

(SEQ ID NO: 34)
CAGCACCAGCTCCCACCCCAGCCTCTGCCAGTCTCTTGGAGGTGGTG
TGGGTGCAGACACCGGCTCACAAGGTGCCCTGTTAGTGAGCTTGGGG
TGCCATGGGAGCCTTCT

SEQ ID NO: 35 is an example of a region where a branch point that can be involved in pseudo-exon-type aberrant splicing is present in the APC gene, and represents the base sequence of a region (8) from 112790393rd to 112790503rd bases (82896th to 83006th bases in SEQ ID NO: 3) in the base sequence of human chromosome 5 in the GRCh38/hg38 human reference genome list.

(SEQ ID NO: 35)
TCAGCTCACTGCAACCTCTGCCTCCTGGGTTCGAGCGATTCTCCTGC
CTTAGCCTCCCGAGTAGCTGGGATTACAGGCACGCGTCACCCATGCC
TGGCTAATTTCTTTTTG

SEQ ID NO: 36 is another example of a region where a branch point that can be involved in pseudo-exon-type aberrant splicing is present in the APC gene, and represents the base sequence of a region (9) from 112822518th to 112822628th bases (115021st to 115131st bases in SEQ ID NO: 3) in the base sequence of human chromosome 5 in the GRCh38/hg38 human reference genome list.

(SEQ ID NO: 36)
AAACATTTCATTATAACTTTGGAATGATTACTTCTTTAGGTATGTAT
TTTCACACACTAAGCCTTATATAACCAGCAGTGCACTCCATTTTTTA
TGTAATGGTTTTTCTTT

SEQ ID NO: 37 is still another example of a region where a branch point that can be involved in pseudo-exon-type aberrant splicing is present in the APC gene, and represents the base sequence of a region (10) from 112779563rd to 112779673rd bases (72067th to 72177th bases in SEQ ID NO: 3) in the base sequence of human chromosome 5 in the GRCh38/hg38 human reference genome list.

(SEQ ID NO: 37)
AGCCAAGCTAATGAACACTTTATGTGGAAATACCTACTTATCAAAAC
ATTACTGAAAACATCAGATCTAAACCACATTATTGCAACATACTGTG
TCATGTTCAATTTTTTT

SEQ ID NO: 38 is an example of a region where a branch point that can be involved in pseudo-exon-type aberrant splicing is present in the ATM gene, and represents the base sequence of a region (11) from 108270351st to 108270461st bases (47285th to 47395th bases in SEQ ID NO: 4) in the base sequence of human chromosome 11 in the GRCh38/hg38 human reference genome list.

(SEQ ID NO: 38)
GAGACTTACAGTTTCAGAATCTTGCTCAAGCTCTTAACTGCAACAGT
GGTAATAATGATCATTTATTGAATTCCACAATAGAAGCTAGACTTTT
ACGTTTATTTTCTCTAA

SEQ ID NO: 39 is another example of a region where a branch point that can be involved in pseudo-exon-type aberrant splicing is present in the ATM gene, and represents the base sequence of a region (12) from 108308849th to 108308959th bases (85783rd to 85893rd bases in SEQ ID NO: 4) in the base sequence of human chromosome 11 in the GRCh38/hg38 human reference genome list.

(SEQ ID NO: 39)
ACCTTAGAGTTTTATACCAGATTATCTTCTGAGGAGGCCTATCAGAA
GCTTTAATGTAGTGGAGAGCATTTGTTTTCTTGGTGTTGGGAGGCAG
TTTTACCTTTGAGTCAT

SEQ ID NO: 40 is an example of a region where a branch point that can be involved in pseudo-exon-type aberrant splicing is present in the BRCA1 gene, and represents the base sequence of a region (13) from 43087074th to 43086964th bases (38291st to 38401st bases in SEQ ID NO: 5) in the base sequence of human chromosome 17 in the GRCh38/hg38 human reference genome list.

(SEQ ID NO: 40)
TCCCATTTTCCTGTACCTTGCCAACACTGGGTGATATCCAGTTTTAA
AATCTAAATCTTGCATTGCTATGAGAACTACAATTAGAGAAGGCTTA
TCTTCTACTGCCCATTC

SEQ ID NO: 41 is an example of a region where a branch point that can be involved in pseudo-exon-type aberrant splicing is present in the BRCA2 gene, and represents the base sequence of a region (14) from 32345028th to 32345138th bases (29521st to 29631st bases in SEQ ID NO: 6) in the base sequence of human chromosome 13 in the GRCh38/hg38 human reference genome list.

(SEQ ID NO: 41)
AAGTAGTAGAAAGCTGTCAAGCTTACAGAGCCAGATACAAGCTTCCCAA
AAATTCTGATTTTCATCTAAAAGCTTGAATTTTTCCCCGGCAATAAGTA
TTGTCACTTATTT

SEQ ID NO: 42 is an example of a region where a branch point that can be involved in pseudo-exon-type aberrant splicing is present in the BRIP1 gene, and represents the base sequence of a region (15) from 61/782,587th to 61/782,477th bases (80942nd to 81052nd bases in SEQ ID NO: 7) in the base sequence of human chromosome 17 in the GRCh38/hg38 human reference genome list.

(SEQ ID NO: 42)
CTCTCATTCTGTGGGTTGTTTTTTCATTTATTCATAGTGTCCTTTGATG
CAGAAAAGGTTTTTAATCTTGTTGAAGTCCAATTTATCTTTCTCTTTTC
TTGTTGTTGCCTG

SEQ ID NO: 43 is an example of a region where a branch point that can be involved in pseudo-exon-type aberrant splicing is present in the CEP290 gene, and represents the base sequence of a region (16) from 88101435th to 88101325th bases (40654th to 40764th bases in SEQ ID NO: 8) in the base sequence of human chromosome 12 in the GRCh38/hg38 human reference genome list.

(SEQ ID NO: 43)
CATCTTGGCTCACTGCAAGCTCCACCTCCCGGGTTCAGGCCGTTCTCCT
GCCTCAGCCTCCTGAGTAGCTGGTACCACAGGCACCCACCATCATGCCC
GGCTAATTTTTTG

SEQ ID NO: 44 is an example of a region where a branch point that can be involved in pseudo-exon-type aberrant splicing is present in the CFTR gene, and represents the base sequence of a region (17) from 117578099th to 117578209th bases (98075th to 98185th bases in SEQ ID NO: 9) in the base sequence of human chromosome 7 in the GRCh38/hg38 human reference genome list.

(SEQ ID NO: 44)
ATTCAATTGTATATGTGTATATAGCCAAGTTATTGTACAGTTGACCTTT
GAACAACACGGGTTTGAACTATGCAGGTCCACTTACACGTATTTTTTTT
TTCCGTTTCTGAC

SEQ ID NO: 45 is another example of a region where a branch point that can be involved in pseudo-exon-type aberrant splicing is present in the CFTR gene, and represents the base sequence of a region (18) from 117589298th to 117589408th bases (109274th to 109384th bases in SEQ ID NO: 9) in the base sequence of human chromosome 7 in the GRCh38/hg38 human reference genome list.

(SEQ ID NO: 45)
AGTTACACTATAAAGGTTGTTTTAGACTTTTAAAGTTTTGCCATTGGTT
TTTAAAAAAATTTTTAAATTGGCTTTAAAAATTTCTTAATTGTGTGCTG
AATACAATTTTCT

SEQ ID NO: 46 is still another example of a region where a branch point that can be involved in pseudo-exon-type aberrant splicing is present in the CFTR gene, and represents the base sequence of a region (19) from 117639756th to 117639866th bases (159732nd to 159842nd bases in SEQ ID NO: 9) in the base sequence of human chromosome 7 in the GRCh38/hg38 human reference genome list.

(SEQ ID NO: 46)
TTGAATCATTCAGTGGGTATAAGCAGCATATTCTCAATACTATGTTTCA
TTAATAATTAATAGAGATATATGAACACATAAAAGATTCAATTATAATC
ACCTTGTGGATCT

SEQ ID NO: 47 is an example of a region where a branch point that can be involved in pseudo-exon-type aberrant splicing is present in the CLRN1 gene, and represents the base sequence of a region (20) from 150942759th to 150942649th bases (30241st to 30351st bases in SEQ ID NO: 10) in the base sequence of human chromosome 3 in the GRCh38/hg38 human reference genome list.

(SEQ ID NO: 47)
TTAAATGAGAAGTGACACATGCGTAAAAGGGGGAAAAGCATTGATTGTG
GCCATTTTTGGAGATAAGCTATCACGTTTATTTGTTTGTTTGCTTTCTA
ATGGTCTGTCTTC

SEQ ID NO: 48 is an example of a region where a branch point that can be involved in pseudo-exon-type aberrant splicing is present in the CNGB3 gene, and represents the base sequence of a region (21) from 86605573rd to 86605463rd bases (138062nd to 138172nd bases in SEQ ID NO: 11) in the base sequence of human chromosome 8 in the GRCh38/hg38 human reference genome list.

(SEQ ID NO: 48)
TAGTGTATTCTAATTCTTTCAAAGTATGGTTAATGAGAATATTTGATTA
ACTCAAATAACCCAGTCCCCTCCTAAGCCAAGTAAGTGAATTTATTGTA
TTAATGCTATTTT

SEQ ID NO: 49 is an example of a region where a branch point that can be involved in pseudo-exon-type aberrant splicing is present in the COL6A1 gene, and represents the base sequence of a region (22) from 45989774th to 45989884th bases (8005th to 8115th bases in SEQ ID NO: 12) in the base sequence of human chromosome 21 in the GRCh38/hg38 human reference genome list.

(SEQ ID NO: 49)
AGAAGGTGAGTGAGGCTCGACCTCGGAGCTGGTCTCTCCAGGCGCAGAT
GTGCCATCCTGGACGAGGGTGTCCCCGGGGATGAGGACAGTGTCCCTGA
CAGGAGACCACGT

SEQ ID NO: 50 is an example of a region where a branch point that can be involved in pseudo-exon-type aberrant splicing is present in the DYSF gene, and represents the base sequence of a region (23) from 71661602nd to 71661712th bases (208042nd to 208152nd bases in SEQ ID NO: 13) in the base sequence of human chromosome 2 in the GRCh38/hg38 human reference genome list.

(SEQ ID NO: 50)
TGCCCATTGCATGGGAGTAATTCCTAGGCATCCTGAATTGCTGTTTGGA
TGTGAGCTTGTTTAGGCCAGAGAGGGGAGGATGCAGAGGGAGGGTGGCA
GCTATTTCTCTCG

SEQ ID NO: 51 is an example of a region where a branch point that can be involved in pseudo-exon-type aberrant splicing is present in the FKTN gene, and represents the base sequence of a region (24) from 105606387th to 105606497th bases (48258th to 48368th bases in SEQ ID NO: 14) in the base sequence of human chromosome 9 in the GRCh38/hg38 human reference genome list.

(SEQ ID NO: 51)
TTTGATTAGCTATAGTTTATAATATTAACAATTACAGGATTAAAAACAT
TCTTGAAGTTATACTTGGAGTATGAAGTTTCTAACCTAGAATTGTTCCT
TTTATTCTCCTTT

SEQ ID NO: 52 is an example of a region where a branch point that can be involved in pseudo-exon-type aberrant splicing is present in the GAA gene, and represents the base sequence of a region (25) from 80104281st to 80104391st bases (2701st to 2811th bases in SEQ ID NO: 15) in the base sequence of human chromosome 17 in the GRCh38/hg38 human reference genome list.

(SEQ ID NO: 52)
GCTGGTCTTCCTGGGGACATTCTAAGCGTGTTTGATTTGTAACATTTTA
GCAGACTGTGCAAGTGCTCTGCACTCCCCTGCTGGAGCTTTTCTCGCCC
TTCCTTCTGGCCC

SEQ ID NO: 53 is an example of a region where a branch point that can be involved in pseudo-exon-type aberrant splicing is present in the GALT gene, and represents the base sequence of a region (26) from 34649835th to 34649945th bases (3161st to 3271st bases in SEQ ID NO: 16) in the base sequence of human chromosome 9 in the GRCh38/hg38 human reference genome list.

(SEQ ID NO: 53)
GCTAAACTCTTTCATCCCCTGGTGGCTTCAGCAGTCCTTATCACCAGCC
TCACAATCCCACAGGCCCACCCCCAGTGGGCCTGTGGCATTCATATTTC
ATATTCATATTTC

SEQ ID NO: 54 is an example of a region where a branch point that can be involved in pseudo-exon-type aberrant splicing is present in the GHR gene, and represents the base sequence of a region (27) from 42700567th to 42700677th bases (277129th to 277239th bases in SEQ ID NO: 17) in the base sequence of human chromosome 5 in the GRCh38/hg38 human reference genome list.

(SEQ ID NO: 54)
TCCTTACCAAGCATATGGAACTCAGCATTTTGATAAATTTCACATGGCA
CATAACAAGAGGAAAAACAGGAGTATCATGCTGCTCCCAATATAACTAA
TTCTAAATCTGTC

SEQ ID NO: 55 is an example of a region where a branch point that can be involved in pseudo-exon-type aberrant splicing is present in the GLA gene, and represents the base sequence of a region (28) from 101399920th to 101399810th bases (8006th to 8116th bases in SEQ ID NO: 18) in the base sequence of human chromosome X in the GRCh38/hg38 human reference genome list.

(SEQ ID NO: 55)
CTTAACATTGAAGTCGCAGACCAAACGCCACATATGCAGACAGTTCTTC
TCTAACTACTTTAAAATAGCCCTCTGTCCATTCATTCTTCATCACATTA
ACCTGTTTAATTT

SEQ ID NO: 56 is an example of a region where a branch point that can be involved in pseudo-exon-type aberrant splicing is present in the HADH gene, and represents the base sequence of a region (29) from 108023829th to 108023939th bases (33941st to 34051st bases in SEQ ID NO: 19) in the base sequence of human chromosome 4 in the GRCh38/hg38 human reference genome list.

(SEQ ID NO: 56)
GACTGCTCTCTGTCCTGGCTTTTGTCTCCTGATGAATGGCTGCATTTTC
ATAAATGATTTTAGGTACAGCTTGGTAAACACATACCTCCCTAACAGAA
AATGAGGGCTTTA

SEQ ID NO: 57 is an example of a region where a branch point that can be involved in pseudo-exon-type aberrant splicing is present in the HBB gene, and represents the base sequence of a region (30) from 5226117th to 5226007th bases (955th to 1065th bases in SEQ ID NO: 20) in the base sequence of human chromosome 11 in the GRCh38/hg38 human reference genome list.

(SEQ ID NO: 57)
TACACATATTGACCAAATCAGGGTAATTTTGCATTTGTAATTTTAAAAA
ATGCTTTCTTCTTTTAATATACTTTTTTGTTTATCTTATTTCTAATACT
TTCCCTAATCTCT

SEQ ID NO: 58 is an example of a region where a branch point that can be involved in pseudo-exon-type aberrant splicing is present in the LDLR gene, and represents the base sequence of a region (31) from 11120250th to 11120360th bases (30788th to 30898th bases in SEQ ID NO: 21) in the base sequence of human chromosome 19 in the GRCh38/hg38 human reference genome list.

(SEQ ID NO: 58)
CCCACCCCCCCAACCTTGAAACCTCCTTGTGGAAACTCTGGAATGTTCT
GGAAATTTCTGGAATCTTCTGGTATAGCTGATGATCTCGTTCCTGCCCT
GACTCCGCTTCTT

SEQ ID NO: 59 is another example of a region where a branch point that can be involved in pseudo-exon-type aberrant splicing is present in the LDLR gene, and represents the base sequence of a region (32) from 11122702nd to 11122812th bases (33240th to 33350th bases in SEQ ID NO: 21) in the base sequence of human chromosome 19 in the GRCh38/hg38 human reference genome list.

(SEQ ID NO: 59)
TGAGCCACCTCGCCCAGCCTGAGCCACCTCACCCAGCCTAAGCCACTGT
GCCTGGCCTGATTTTGGACTTTTTAAAAATTTTATTAATAATTATTTTT
GGGTTTCTTTTTT

SEQ ID NO: 60 is an example of a region where a branch point that can be involved in pseudo-exon-type aberrant splicing is present in the MYBPC3 gene, and represents the base sequence of a region (33) from 47343432nd to 47343322nd bases (9271st to 9381st bases in SEQ ID NO: 22) in the base sequence of human chromosome 11 in the GRCh38/hg38 human reference genome list.

(SEQ ID NO: 60)
TCCTGCCCCTCTCCACATGCGTATCTCTGACTCGGTGTGGCTCTCAGCC
CCATCTCTCTGGGCCTAATTTCCCATCCTTTTGCTCCTGCCGGTCCCTC
TCTCTCTCTCCTT

SEQ ID NO: 61 is an example of a region where a branch point that can be involved in pseudo-exon-type aberrant splicing is present in the PKHD1 gene, and represents the base sequence of a region (34) from 51882676th to 51882566th bases (204940th to 205050th bases in SEQ ID NO: 23) in the base sequence of human chromosome 6 in the GRCh38/hg38 human reference genome list.

(SEQ ID NO: 61)
GATGGGGCAAAGAGTACTCCCGGGTGGGAAGCACTAGTTCCTAAGTGGG
GGTTCCTGGCTGCCATTGGTCTCTTAGGCTTCAGGTCTATAATGGATCC
CCTATGTTCTTGT

SEQ ID NO: 62 is an example of a region where a branch point that can be involved in pseudo-exon-type aberrant splicing is present in the RPGRIP1 gene, and represents the base sequence of a region (35) from 21320889th to 21320999th bases (40807th to 40917th bases in SEQ ID NO: 24) in the base sequence of human chromosome 14 in the GRCh38/hg38 human reference genome list.

(SEQ ID NO: 62)
CTGTGCCCTGCTTGAGGACACTTTTTGGAAAACTGTGAGAAGGCAGAGC
GTAGAGAACTTCATGAGCTCCACCCATTTCTTCCACTCTTTGCAGCTCA
TAAAATTTAGAAT

SEQ ID NO: 63 is an example of a region where a branch point that can be involved in pseudo-exon-type aberrant splicing is present in the SLC12A3 gene, and represents the base sequence of a region (36) from 56883499th to 56883609th bases (18293rd to 18403rd bases in SEQ ID NO: 25) in the base sequence of human chromosome 16 in the GRCh38/hg38 human reference genome list.

(SEQ ID NO: 63)
CTCGATCTCCTGACCTCATGATCCGCCCGCCTCAGCCTCCCAAAGTGCT
GGGATTACAGTTGTGCCTGGCTGAGGAAGACTTTTTCTAACCAGCTCCA
AATTGCCATTGTC

SEQ ID NO: 64 is another example of a region where a branch point that can be involved in pseudo-exon-type aberrant splicing is present in the SLC12A3 gene, and represents the base sequence of a region (37) from 56893096th to 56893206th bases (27890th to 28000th bases in SEQ ID NO: 25) in the base sequence of human chromosome 16 in the GRCh38/hg38 human reference genome list.

(SEQ ID NO: 64)
CGGGGTGGTGGTGGTCTTCCTTCCTTCTCCTTCCTGGCCTGCTCTCAAA
GGGGACAGGGGCTCCTGGGCCCAGCAGTGAGCTCAGGGGAGCCCAGAGG
GACCCCTCTGTCT

SEQ ID NO: 65 is an example of a region where a branch point that can be involved in pseudo-exon-type aberrant splicing is present in the USH2A gene, and represents the base sequence of a region (38) from 215891470th to 215891360th bases (531979th to 532089th bases in SEQ ID NO: 26) in the base sequence of human chromosome 1 in the GRCh38/hg38 human reference genome list.

(SEQ ID NO: 65)
TTGATTTGTATATAGAATTAGATGATTCGGCTTATCATTTTAAAGCACT
AAATTGAAAGAGTGCCAGGAGTCAGGTTTTAACACTTCCCTAGCCAAAG
GAGCTAATTAAGC

SEQ ID NO: 66 is another example of a region where a branch point that can be involved in pseudo-exon-type aberrant splicing is present in the USH2A gene, and represents the base sequence of a region (39) from 215794716th to 215794606th bases (628733rd to 628843rd bases in SEQ ID NO: 26) in the base sequence of human chromosome 1 in the GRCh38/hg38 human reference genome list.

(SEQ ID NO: 66)
TAGAGTTTCTCATGGCATTCTACAGCTCTGCCAACTCTGATAATCATAG
TGGACTTAAGGAATAATAGTTTTACAAAGGAAAAAATATATCTTTTTAT
TCTCTTGACTTTC

SEQ ID NO: 67 is an example of a region where a branch point that can be involved in pseudo-exon-type aberrant splicing is present in the WRN gene, and represents the base sequence of a region (40) from 31108365th to 31108475th bases (74556th to 74666th bases in SEQ ID NO: 27) in the base sequence of human chromosome 8 in the GRCh38/hg38 human reference genome list.

(SEQ ID NO: 67)
CACTCTAGTTTATATATTTTAAATGTCATAAAATACCACATACTTATAA
GAGAAAAGGTTCTATTCATTGCTGAAGTGGAAGCTTATCATTAATTTTT
ATTTATTTATTTT

In one or more non-limiting embodiments, the antisense oligonucleotide of the present disclosure is an antisense oligonucleotide including the base sequence represented by SEQ ID NO: 68, an antisense oligonucleotide including the base sequence represented by SEQ ID NO: 69, or an antisense oligonucleotide including the base sequence represented by SEQ ID NO: 70, and is preferably an antisense oligonucleotide consisting of the base sequence represented by SEQ ID NO: 68, an antisense oligonucleotide consisting of the base sequence represented by SEQ ID NO: 69, or an antisense oligonucleotide consisting of the base sequence represented by SEQ ID NO: 70.

In one or more embodiments, examples of the antisense oligonucleotide of the present disclosure include an antisense oligonucleotide having a base sequence of any of SEQ ID NOs: 68 to 70 with deletion, substitution, insertion, and/or addition of 1 to 5 bases, 1 to 4 bases, 1 to 3 bases, 1 or 2 bases, or 1 base and having activity capable of suppressing and/or correcting a pseudo-exon-type splicing defect caused by a mutation in the FKTN gene (c.647+2084G>T (chr9:105606576G>T)).

In one or more embodiments, examples of the antisense oligonucleotide of the present disclosure include an antisense oligonucleotide having a base sequence with at least 80%, at least 85%, at least 90%, at least 91%, at least 92%, at least 93%, at least 94%, at least 95%, at least 96%, at least 97%, at least 98%, at least 99%, or 100% sequence identity to any of the base sequence of SEQ ID NOs: 68 to 70 and having activity capable of suppressing and/or correcting a pseudo-exon-type splicing defect caused by the mutation in the FKTN gene (c.647+2084G>T (chr9:105606576G>T)).

In one or more embodiments, examples of the antisense oligonucleotide of the present disclosure include an antisense oligonucleotide having a base sequence represented by SEQ ID NO: 68 with deletion, substitution, insertion, and/or addition of 1 to 5 bases, 1 to 4 bases, 1 to 3 bases, 1 or 2 bases, or 1 base and capable of binding to its target sequence (the base sequence consisting of bases at positions 48321 to 48345 of SEQ ID NO: 14).

In one or more embodiments, examples of the antisense oligonucleotide of the present disclosure include an antisense oligonucleotide having a base sequence represented by SEQ ID NO: 69 with deletion, substitution, insertion, and/or addition of 1 to 5 bases, 1 to 4 bases, 1 to 3 bases, 1 or 2 bases, or 1 base and capable of binding to its target sequence (the base sequence consisting of bases at positions 48337 to 48361 of SEQ ID NO: 14).

In one or more embodiments, examples of the antisense oligonucleotide of the present disclosure include an antisense oligonucleotide having a base sequence represented by SEQ ID NO: 70 with deletion, substitution, insertion, and/or addition of 1 to 5 bases, 1 to 4 bases, 1 to 3 bases, 1 or 2 bases, or 1 base and capable of binding to its target sequence (the base sequence consisting of bases at positions 48349 to 48368 of SEQ ID NO: 14).

In one or more embodiments, examples of the antisense oligonucleotide of the present disclosure include an antisense oligonucleotide having a base sequence with at least 80%, at least 85%, at least 90%, at least 91%, at least 92%, at least 93%, at least 94%, at least 95%, at least 96%, at least 97%, at least 98%, at least 99%, or 100% sequence identity to the base sequence represented by SEQ ID NO: 68 and capable of binding to its target sequence (the base sequence consisting of bases at positions 48321 to 48345 of SEQ ID NO: 14).

In one or more embodiments, examples of the antisense oligonucleotide of the present disclosure include an antisense oligonucleotide having a base sequence with at least 80%, at least 85%, at least 90%, at least 91%, at least 92%, at least 93%, at least 94%, at least 95%, at least 96%, at least 97%, at least 98%, at least 99%, or 100% sequence identity to the base sequence represented by SEQ ID NO: 69 and capable of binding to its target sequence (the base sequence consisting of bases at positions 48337 to 48361 of SEQ ID NO: 14).

In one or more embodiments, examples of the antisense oligonucleotide of the present disclosure include an antisense oligonucleotide having a base sequence with at least 80%, at least 85%, at least 90%, at least 91%, at least 92%, at least 93%, at least 94%, at least 95%, at least 96%, at least 97%, at least 98%, at least 99%, or 100% sequence identity to the base sequence represented by SEQ ID NO: 70 and capable of binding to its target sequence (the base sequence consisting of bases at positions 48349 to 48368 of SEQ ID NO: 14).

In one or more non-limiting embodiments, the antisense oligonucleotide of the present disclosure is an antisense oligonucleotide including the base sequence represented by SEQ ID NO: 74, and is preferably an antisense oligonucleotide consisting of the base sequence represented by SEQ ID NO: 74.

In one or more embodiments, examples of the antisense oligonucleotide of the present disclosure include an antisense oligonucleotide having a base sequence of SEQ ID NO: 74 with deletion, substitution, insertion, and/or addition of 1 to 5 bases, 1 to 4 bases, 1 to 3 bases, 1 or 2 bases, or 1 base and having activity capable of suppressing and/or correcting a pseudo-exon-type splicing defect caused by a mutation in the CFTR gene (c.3849+12191C>T (chr7:117639961C>T)).

In one or more embodiments, examples of the antisense oligonucleotide of the present disclosure include an antisense oligonucleotide having a base sequence with at least 80%, at least 85%, at least 90%, at least 91%, at least 92%, at least 93%, at least 94%, at least 95%, at least 96%, at least 97%, at least 98%, at least 99%, or 100% sequence identity to the base sequence of SEQ ID NO: 74 and having activity capable of suppressing and/or correcting a pseudo-exon-type splicing defect caused by the mutation in the CFTR gene (c.3849+12191C>T (chr7:117639961C>T)).

In one or more embodiments, examples of the antisense oligonucleotide of the present disclosure include DNA strands, RNA strands, and DNA-RNA hybrid strands. In one or more embodiments, the antisense oligonucleotide of the present disclosure may be an oligodeoxyribonucleotide, an oligoribonucleotide, a chimeric oligonucleotide composed of a deoxyribonucleotide and a ribonucleotide, or the like.

In one or more embodiments, the antisense oligonucleotide of the present disclosure may include a chemically modified oligonucleotide. In one or more embodiments, examples of chemical modification include modification of phosphorus atoms (substitution of oxygen atoms in diester linkage of phosphate moieties) and modification (conjugation) in nucleoside sugar moieties. In one or more embodiments, examples of the modification of phosphorus atoms include phosphorothioate modification, boranophosphate modification, methylphosphonate modification, and phosphorodithioate modification. In one or more embodiments, examples of the modification in nucleoside sugar moieties include ribose 2′modifications, such as 2′-O-methoxyethyl (2′-MOE), 2′-O-methyl (2′-OMe), and 2′-fluoro (2′-F).

In one or more embodiments, the antisense oligonucleotide of the present disclosure may include a nucleotide analogue and/or a spacer. In one or more embodiments, examples of the nucleotide analogue include morpholino backbones, carbamate backbones, siloxane backbones, sulfide backbones, sulfoxide backbones, sulfone backbones, formacetyl backbones, thioformacetyl backbones, methylene formacetyl backbones, riboacetyl backbone, alkene-containing backbones, sulfamate backbones, sulfonate backbones, sulfonamide backbones, methyleneimino backbones, methylenehydrazino backbones, and amide backbones.

In one or more embodiments, the antisense oligonucleotide of the present disclosure can be produced using a known genetic engineering method, chemical synthesis method, or the like.

### Method for Producing Antisense Oligonucleotide

Still another aspect of the present disclosure relates to a method for producing an antisense oligonucleotide for correcting pseudo-exon-type aberrant splicing. The antisense oligonucleotide production method of the present disclosure includes producing a base sequence complementary to a target sequence that includes a branch point in pseudo-exon-type aberrant splicing and consists of 10 to 50 bases.

In one or more embodiments, the antisense oligonucleotide production method of the present disclosure may include: measuring, with respect to obtained base sequences, activity for suppressing pseudo-exon-type aberrant splicing in which the branch point included in the target sequence is involved and/or efficiency of suppressing the pseudo-exon-type aberrant splicing; and based on the thus-measured activities and/or efficiencies, selecting a base sequence (antisense oligonucleotide) exhibiting the activity and/or efficiency exceeding a predetermined threshold value.

In one or more embodiments, the antisense oligonucleotide production method of the present disclosure may include determining a target sequence that includes a branch point in pseudo-exon-type aberrant splicing. The target sequence used in the production method of the present disclosure includes a branch point in pseudo-exon-type aberrant splicing. In one or more embodiments, the target sequence used in the production method of the present disclosure is a base sequence that includes a branch point in pseudo-exon-type aberrant splicing and consists of 10 to 50 successive bases in any of the above-described regions (1) to (40). In one or more embodiments, examples of the branch point include those described above.

In one or more embodiments, the length of the target sequence is 10 to 50 bases. In one or more embodiments, the length of the target sequence is 11 bases or more, 12 bases or more, 13 bases or more, 14 bases or more, or 15 bases or more. In one or more embodiments, the length of the antisense oligonucleotide is 45 bases or less, 40 bases or less, 39 bases or less, 38 bases or less, 37 bases or less, 36 bases or less, 35 bases or less, 34 bases or less, 33 bases or less, 32 bases or less, 30 bases or less, 29 bases or less, 28 bases or less, 27 bases or less, 26 bases or less, or 25 bases or less.

In one or more embodiments, the base sequence complementary to the target sequence need not necessarily be perfectly complementary to the target sequence. In one or more embodiments, the base sequence complementary to the target sequence may include mismatches to the extent that the base sequence can form a hybrid with the target sequence and can suppress pseudo-exon-type aberrant splicing in which the branch point included in the target sequence is involved. In one or more embodiments, the number of mismatch bases that may be included in the base sequence is 1, 2, 3, 4, 5, 6, 7, 8, 9, or 10.

In one or more embodiments, the base sequence complementary to the target sequence need only be such that at least 40% of bases of the base sequence to be produced are complementary to the target sequence, and preferably at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of bases of the base sequence to be produced are complementary to the target sequence.

In one or more embodiments, the length of the base sequence (antisense oligonucleotide) produced by the production method of the present disclosure is 10 bases to 50 bases. In one or more embodiments, the length of the antisense oligonucleotide is 11 bases or more, 12 bases or more, 13 bases or more, 14 bases or more, or 15 bases or more. In one or more embodiments, the length of the antisense oligonucleotide is 45 bases or less, 40 bases or less, 39 bases or less, 38 bases or less, 37 bases or less, 36 bases or less, 35 bases or less, 34 bases or less, 33 bases or less, 32 bases or less, 30 bases or less, 29 bases or less, 28 bases or less, 27 bases or less, 26 bases or less, or 25 bases or less. In one or more non-limiting embodiments, the length of the antisense oligonucleotide is preferably 15 bases to 30 bases.

In one or more non-limiting embodiments, examples of a base sequence obtained in the step of producing the base sequence complementary to the target sequence include a base sequence having 1 to 5 mismatch bases relative to the target sequence excluding the branch point and predicted to bind to less than 20 off-targets. The base sequence may be a base sequence having 1 to 4 mismatch bases and predicted to bind to less than 10 off-targets, or a base sequence having 1 to 3 mismatch bases and predicted to bind to 5 or less off-targets. In one or more embodiments, the length of such a base sequence is 15 bases to 30 bases and is preferably 25 bases.

In one or more embodiments, the antisense oligonucleotide production method of the present disclosure may include determining a target sequence. In one or more embodiments, the antisense oligonucleotide production method of the present disclosure may include determining a branch point to be included in a target sequence and determining the target sequence based on the thus-determined branch point. In one or more embodiments, the target sequence can be designed based on a disease of interest and/or the base sequence of a genomic DNA, cDNA, pre-mRNA, or mRNA of a gene.

In one or more non-limiting embodiments, the production method of the present disclosure can produce an antisense oligonucleotide for correcting pseudo-exon-type splicing defects caused by the above-described mutations (variants). The branch point included in the target sequence is as described above.

### Pharmaceutical Composition

Still another aspect of the present disclosure relates to a pharmaceutical composition containing the antisense oligonucleotide of the present disclosure as an active ingredient. In one or more embodiments, the pharmaceutical composition of the present disclosure is capable of correcting pseudo-exon-type aberrant splicing. In one or more embodiments, the pharmaceutical composition of the present disclosure can be used for preventing, treating, and/or ameliorating the development of a disease caused by pseudo-exon-type aberrant splicing. In one or more embodiments, examples of the disease caused by pseudo-exon-type aberrant splicing include those described above.

In the present disclosure, “treatment” refers to alleviating and/or removing a disease, illness, or disorder that a subject already has or symptoms caused thereby. In the present disclosure, “prevention of the development” refers to preventing a subject from having a disease, illness, or disorder.

In one or more embodiments, the pharmaceutical composition of the present disclosure contains the antisense oligonucleotide of the present disclosure, and may further contain a pharmaceutically acceptable carrier, antiseptic agent, diluent, excipient, and/or any other pharmaceutically acceptable ingredient.

In one or more embodiments, the content of the antisense oligonucleotide of the present disclosure as the active ingredient in the pharmaceutical composition of the present disclosure can be determined as appropriate according to the dosage form, administration method, carrier, and the like. In one or more embodiments, the amount of the antisense oligonucleotide of the present disclosure with respect to the total amount of formulation may be 0.01% to 100% (w/w), 0.1% to 95% (w/w), or the like.

In one or more embodiments, the pharmaceutical composition of the present disclosure can be processed into a dosage form suitable for an administration form by applying a well-known formulation technique. In one or more embodiments, examples of the administration form include oral administration and parenteral administration. In one or more embodiments, the formulation for oral administration may have a dosage form such as a tablet, capsule, granule, powder medicine, pill, troche, syrup, or a liquid medicine (e.g., a solution or suspension). In one or more embodiments, the formulation for parenteral administration may be an injectable, aerosol, or the like. In one or more embodiments, these formulations can be produced by a well-known method using additives. In one or more embodiments, examples of the additives include excipients, lubricants, binders, disintegrants, stabilizers, taste and odor masking agents, and diluents.

In one or more embodiments, examples of the excipients include various starches such as starch, potato starch, and corn starch, lactose, crystalline celluloses, and calcium hydrogen phosphate. In one or more embodiments, examples of the lubricants include ethyl cellulose, shellac, talc, carnauba wax, and paraffin. In one or more embodiments, examples of the binders include polyvinylpyrrolidone, macrogol, hydroxypropyl cellulose, hydroxypropyl methylcellulose, and the same compounds as those given as examples of the excipients. In one or more embodiments, examples of the disintegrants include the same compounds as those given as examples of the excipients, as well as chemically modified starches and celluloses, such as croscarmellose sodium, sodium carboxymethyl starch, and cross-linked polyvinylpyrrolidone. In one or more embodiments, examples of the stabilizer include: p-hydroxybenzoic acid esters such as methylparaben and propylparaben; alcohols such as chlorobutanol, benzyl alcohol, and phenylethyl alcohol; benzalkonium chloride; various phenols such as phenol and cresol; thimerosal; dehydroacetic acid; and sorbic acid. In one or more embodiments, examples of the taste and odor masking agents include commonly used sweeteners, acidulants, and flavors.

In one or more embodiments, a liquid medicine for oral administration can be produced using, for example, ethanol, phenol, chlorocresol, purified water, and/or distilled water as a solvent, and when necessary, a surfactant, preservative, tonicity agent, pH adjuster, emulsifying agent, and/or the like can also be used. In one or more embodiments, the liquid medicine for oral administration may further contain a solubilizer, wetting agent, suspending agent, sweetening agent, taste masking agent, aromatic agent, and antiseptic agent.

An injectable for parenteral administration may be a sterile aqueous or non-aqueous liquid, suspension, or emulsion. In one or more embodiments, an aqueous solvent for the injectable may be distilled water or physiological saline. In one or more embodiments, a non-aqueous solvent for the injectable may be a vegetable oil, an alcohol, and/or polysorbate 80 (pharmacopeia name). Examples of the vegetable oil include propylene glycol, polyethylene glycol, and olive oil. Examples of the alcohol include ethanol. In one or more embodiments, the injectable may further contain a tonicity agent, antiseptic agent, wetting agent, emulsifying agent, dispersant, stabilizer, dissolution assisting agent, and/or the like. In one or more embodiments, such a formulation may be sterilized by filtration through a bacteria-retaining filter, blending of a bactericide, or irradiation. Alternatively, a sterile solid composition may be dissolved or suspended in sterile water or a sterile solvent for injectable before use, and the thus-obtained composition may be used as such a formulation.

In one or more embodiments, the pharmaceutical composition of the present disclosure may be administered in the form of a non-viral vector or viral vector. Besides, a method of introducing the pharmaceutical composition using a liposome, a microinjection method, a method of transferring the antisense oligonucleotide into cells together with a carrier using a gene gun, an ultrasonic introduction method, and the like may be used in combination to administer the pharmaceutical composition of the present disclosure.

The method of using the pharmaceutical composition of the present disclosure may vary depending on symptoms, age, administration method, and the like. In one or more embodiments, the pharmaceutical composition of the present disclosure may be administered intermittently or continuously by oral, transdermal, submucosal, subcutaneous, intramuscular, intravascular, intracerebral, or intraperitoneal administration in such a manner that the body concentration of the antisense oligonucleotide of the present disclosure as the active ingredient would be between 100 pM to 1 mM inclusive. As a non-limiting embodiment, in the case of oral administration, the pharmaceutical composition of the present disclosure may be administered to a subject (an adult in the case of a human) at a dose of 0.01 mg/kg body weight to 2000 mg/kg body weight, 0.1 mg/kg body weight to 500 mg/kg body weight, or 0.1 mg/kg body weight to 100 mg/kg body weight per day in terms of the amount of the antisense oligonucleotide of the present disclosure, either as a single dose or divided into multiple doses, according to the symptoms of the subject. As a non-limiting embodiment, in the case of intravenous administration, the pharmaceutical composition of the present disclosure may be administered to a subject (an adult in the case of a human) at a dose of 0.001 mg/kg body weight to 50 mg/kg body weight or 0.01 mg/kg body weight to 50 mg/kg body weight per day, either as a single dose or divided into multiple doses, according to the symptoms of the subject.

Method for Correcting Pseudo-exon-type Aberrant Splicing Still another aspect of the present disclosure relates to a method for correcting pseudo-exon-type aberrant splicing using an antisense oligonucleotide having a base sequence capable of binding to a sequence including a branch point in pseudo-exon-type aberrant splicing. In one or more embodiments, the correction method of the present disclosure includes administering an effective amount of the antisense oligonucleotide of the present disclosure to a subject in need thereof. In one or more embodiments, the subject may be a patient having a mutation that can cause pseudo-exon-type aberrant splicing. In one or more embodiments, examples of the mutation that can cause pseudo-exon-type aberrant splicing include gene mutations (variants) described above or shown in FIG. 1.

### Treatment Method

Still another aspect of the present disclosure relates to a method for treating a disease caused by pseudo-exon-type aberrant splicing, including administering to a subject an antisense oligonucleotide having a base sequence capable of binding to a sequence that includes a branch point in pseudo-exon-type aberrant splicing. In one or more embodiments, the treatment method of the present disclosure includes administering a therapeutically effective amount of the antisense oligonucleotide of the present disclosure to a patient in need thereof.

In the present disclosure, the “therapeutically effective amount” refers to an amount to be administered to a patient, sufficient for correcting pseudo-exon-type aberrant splicing and/or for treating a disease resulting from pseudo-exon-type aberrant splicing treating.

In one or more embodiments, the subject may be a patient having a mutation that can cause pseudo-exon-type aberrant splicing. In one or more embodiments, examples of the mutation that can cause pseudo-exon-type aberrant splicing include gene mutations (variants) described above or shown in FIG. 1.

In one or more embodiments, examples of the disease caused by pseudo-exon-type aberrant splicing include those described above.

Still another aspect of the present disclosure relates to use of the antisense oligonucleotide of the present disclosure in production of a pharmaceutical composition for treating and/or preventing a disease caused by pseudo-exon-type aberrant splicing.

The present disclosure further relates to one or more non-limiting embodiments to be described below.

[1] An antisense oligonucleotide for correcting pseudo-exon-type aberrant splicing, having a base sequence capable of binding to a sequence that includes a branch point in pseudo-exon-type aberrant splicing.
[2] The antisense oligonucleotide according to [1], wherein
the pseudo-exon-type aberrant splicing is aberrant splicing caused by recognition of a non-coding region as a pseudo-exon, and
the branch point is an adenine base.
   [3] The antisense oligonucleotide according to [1] or [2] for inducing and/or promoting skipping of the pseudo-exon by binding to the sequence including the branch point.
   [4] The antisense oligonucleotide according to any one of [1] to [3], wherein
the branch point is an adenine base located upstream of a 3′-splice site of an intron.
   [5] The antisense oligonucleotide according to any one of [1] to [4], wherein
the branch point is an adenine base located in a region from 10 to 120 bases upstream of a 3′-splice site of an intron.
   [6] The antisense oligonucleotide according to any one of [1] to [5], consisting of 10 to 50 bases.
   [7] An antisense oligonucleotide including a base sequence capable of binding to a target sequence consisting of 10 to 50 successive bases in a region selected from the group consisting of the following regions (1) to (40), an antisense oligonucleotide including a base sequence capable of binding to a target sequence consisting of 10 to 50 successive bases in a region selected from the group consisting of the following regions (1) to (23) and (25) to (40), or an antisense oligonucleotide including a base sequence capable of binding to a target sequence consisting of 10 to 50 successive bases in the following region (19),
wherein the base sequence includes a branch point in pseudo-exon-type aberrant splicing:
   (1) a region that is in an ABCA4 gene and corresponds to a region from 94084339th to 94084229th bases (SEQ ID NO: 28) in a base sequence of human chromosome 1 in a GRCh38/hg38 human reference genome list;
   (2) a region that is in the ABCA4 gene and corresponds to a region from 94081341st to 94081231st bases (SEQ ID NO: 29) in the base sequence of human chromosome 1 in the GRCh38/hg38 human reference genome list;
   (3) a region that is in the ABCA4 gene and corresponds to a region from 94062301st to 94062191st bases (SEQ ID NO: 30) in the base sequence of human chromosome 1 in the GRCh38/hg38 human reference genome list;
   (4) a region that is in the ABCA4 gene and corresponds to a region from 94028532nd to 94028422nd bases (SEQ ID NO: 31) in the base sequence of human chromosome 1 in the GRCh38/hg38 human reference genome list;
   (5) a region that is in the ABCA4 gene and corresponds to a region from 94028576th to 94028466th bases (SEQ ID NO: 32) in the base sequence of human chromosome 1 in the GRCh38/hg38 human reference genome list;
   (6) a region that is in the ABCA4 gene and corresponds to a region from 94027674th to 94027564th bases (SEQ ID NO: 33) in the base sequence of human chromosome 1 in the GRCh38/hg38 human reference genome list;
   (7) a region that is in an ABCC8 gene and corresponds to a region from 17444521st to 17444411th bases (SEQ ID NO: 34) in a base sequence of human chromosome 11 in the GRCh38/hg38 human reference genome list;
   (8) a region that is in an APC gene and corresponds to a region from 112790393rd to 112790503rd bases (SEQ ID NO: 35) in a base sequence of human chromosome 5 in the GRCh38/hg38 human reference genome list;
   (9) a region that is in the APC gene and corresponds to a region from 112822518th to 112822628th bases (SEQ ID NO: 36) in the base sequence of human chromosome 5 in the GRCh38/hg38 human reference genome list;
   (10) a region that is in the APC gene and corresponds to a region from 112779563rd to 112779673rd bases (SEQ ID NO: 37) in the base sequence of human chromosome 5 in the GRCh38/hg38 human reference genome list;
   (11) a region that is in an ATM gene and corresponds to a region from 108270351st to 108270461st bases (SEQ ID NO: 38) in the base sequence of human chromosome 11 in the GRCh38/hg38 human reference genome list;
   (12) a region that is in the ATM gene and corresponds to a region from 108308849th to 108308959th bases (SEQ ID NO: 39) in the base sequence of human chromosome 11 in the GRCh38/hg38 human reference genome list;
   (13) a region that is in a BRCA1 gene and corresponds to a region from 43087074th to 43086964th bases (SEQ ID NO: 40) in a base sequence of human chromosome 17 in the GRCh38/hg38 human reference genome list;
   (14) a region that is in a BRCA2 gene and corresponds to a region from 32345028th to 32345138th bases (SEQ ID NO: 41) in a base sequence of human chromosome 13 in the GRCh38/hg38 human reference genome list;
   (15) a region that is in a BRIP1 gene and corresponds to a region from 61/782,587th to 61/782,477th bases (SEQ ID NO: 42) in the base sequence of human chromosome 17 in the GRCh38/hg38 human reference genome list;
   (16) a region that is in a CEP290 gene and corresponds to a region from 88101435th to 88101325th bases (SEQ ID NO: 43) in a base sequence of human chromosome 12 in the GRCh38/hg38 human reference genome list;
   (17) a region that is in a CFTR gene and corresponds to a region from 117578099th to 117578209th bases (SEQ ID NO: 44) in a base sequence of human chromosome 7 in the GRCh38/hg38 human reference genome list;
   (18) a region that is in the CFTR gene and corresponds to a region from 117589298th to 117589408th bases (SEQ ID NO: 45) in the base sequence of human chromosome 7 in the GRCh38/hg38 human reference genome list;
   (19) a region that is in the CFTR gene and corresponds to a region from 117639756th to 117639866th bases (SEQ ID NO: 46) in the base sequence of human chromosome 7 in the GRCh38/hg38 human reference genome list;
   (20) a region that is in a CLRN1 gene and corresponds to a region from 150942759th to 150942649th bases (SEQ ID NO: 47) in a base sequence of human chromosome 3 in the GRCh38/hg38 human reference genome list;
   (21) a region that is in a CNGB3 gene and corresponds to a region from 86605573rd to 86605463rd bases (SEQ ID NO: 48) in a base sequence of human chromosome 8 in the GRCh38/hg38 human reference genome list;
   (22) a region that is in a COL6A1 gene and corresponds to a region from 45989774th to 45989884th bases (SEQ ID NO: 49) in a base sequence of human chromosome 21 in the GRCh38/hg38 human reference genome list;
   (23) a region that is in a DYSF gene and corresponds to a region from 71661602nd to 71661712th bases (SEQ ID NO: 50) in a base sequence of human chromosome 2 in the GRCh38/hg38 human reference genome list;
   (24) a region that is in a FKTN gene and corresponds to a region from 105606387th to 105606497th bases (SEQ ID NO: 51) in a base sequence of human chromosome 9 in the GRCh38/hg38 human reference genome list;
   (25) a region that is in a GAA gene and corresponds to a region from 80104281st to 80104391st bases (SEQ ID NO: 52) in the base sequence of human chromosome 17 in the GRCh38/hg38 human reference genome list;
   (26) a region that is in a GALT gene and corresponds to a region from 34649835th to 34649945th bases (SEQ ID NO: 53) in the base sequence of human chromosome 9 in the GRCh38/hg38 human reference genome list;
   (27) a region that is in a GHR gene and corresponds to a region from 42700567th to 42700677th bases (SEQ ID NO: 54) in the base sequence of human chromosome 5 in the GRCh38/hg38 human reference genome list;
   (28) a region that is in a GLA gene and corresponds to a region from 101399920th to 101399810th bases (SEQ ID NO: 55) in a base sequence of human chromosome X in the GRCh38/hg38 human reference genome list;
   (29) a region that is in an HADH gene and corresponds to a region from 108023829th to 108023939th bases (SEQ ID NO: 56) in a base sequence of human chromosome 4 in the GRCh38/hg38 human reference genome list;
   (30) a region that is in an HBB gene and corresponds to a region from 5226117th to 5226007th bases (SEQ ID NO: 57) in the base sequence of human chromosome 11 in the GRCh38/hg38 human reference genome list;
   (31) a region that is in an LDLR gene and corresponds to a region from 11120250th to 11120360th bases (SEQ ID NO: 58) in a base sequence of human chromosome 19 in the GRCh38/hg38 human reference genome list;
   (32) a region that is in the LDLR gene and corresponds to a region from 11122702nd to 11122812th bases (SEQ ID NO: 59) in the base sequence of human chromosome 19 in the GRCh38/hg38 human reference genome list;
   (33) a region that is in an MYBPC3 gene and corresponds to a region from 47343432nd to 47343322nd bases (SEQ ID NO: 60) in the base sequence of human chromosome 11 in the GRCh38/hg38 human reference genome list;
   (34) a region that is in a PKHD1 gene and corresponds to a region from 51882676th to 51882566th bases (SEQ ID NO: 61) in a base sequence of human chromosome 6 in the GRCh38/hg38 human reference genome list;
   (35) a region that is in an RPGRIP1 gene and corresponds to a region from 21320889th to 21320999th bases (SEQ ID NO: 62) in a base sequence of human chromosome 14 in the GRCh38/hg38 human reference genome list;
   (36) a region that is in an SLC12A3 gene and corresponds to a region from 56883499th to 56883609th bases (SEQ ID NO: 63) in a base sequence of human chromosome 16 in the GRCh38/hg38 human reference genome list;
   (37) a region that is in the SLC12A3 gene and corresponds to a region from 56893096th to 56893206th bases (SEQ ID NO: 64) in the base sequence of human chromosome 16 in the GRCh38/hg38 human reference genome list;
   (38) a region that is in a USH2A gene and corresponds to a region from 215891470th to 215891360th bases (SEQ ID NO: 65) in the base sequence of human chromosome 1 in the GRCh38/hg38 human reference genome list;
   (39) a region that is in the USH2A gene and corresponds to a region from 215794716th to 215794606th bases (SEQ ID NO: 66) in the base sequence of human chromosome 1 in the GRCh38/hg38 human reference genome list; and
   (40) a region that is in a WRN gene and corresponds to a region from 31108365th to 31108475th bases (SEQ ID NO: 67) in the base sequence of human chromosome 8 in the GRCh38/hg38 human reference genome list.
   [8] A pharmaceutical composition for correcting pseudo-exon-type aberrant splicing, including the antisense oligonucleotide according to any one of [1] to [7] as an active ingredient.
   [9] A pharmaceutical composition for preventing or treating the development of a disease caused by pseudo-exon-type aberrant splicing, including the antisense oligonucleotide according to any one of [1] to [7] as an active ingredient.
   [10] The pharmaceutical composition according to [9], wherein
the disease is at least one selected from the group consisting of Stargardt disease; hyperinsulinemic hypoglycemia, familial; familial adenomatous polyposis; ataxia-telangiectasia; breast-ovarian cancer, familial; breast cancer, early-onset, susceptibility to; Leber congenital amaurosis; cystic fibrosis; Usher syndrome; achromatopsia; Ullrich congenital muscular dystrophy; muscular dystrophy, limb-girdle, autosomal recessive; Fukuyama congenital muscular dystrophy; Pompe disease; galactosemia; Laron dwarfism; Fabry disease; beta-thalassemia; hypercholesterolemia, familial; familial hypertrophic cardiomyopathy; autosomal recessive polycystic kidney disease; Gitelman syndrome; and Werner syndrome.
   [11] A method for correcting pseudo-exon-type aberrant splicing using an antisense oligonucleotide having a base sequence capable of binding to a sequence that includes a branch point in pseudo-exon-type aberrant splicing.
   [12] The method according to [11], wherein
the antisense oligonucleotide is the antisense oligonucleotide according to any one of [1] to [7].
   [13] A method for treating a disease caused by pseudo-exon-type aberrant splicing, including administering to a subject an antisense oligonucleotide having a base sequence capable of binding to a sequence that includes a branch point in pseudo-exon-type aberrant splicing.
   [14] The method according to [13], wherein
the antisense oligonucleotide is the antisense oligonucleotide according to any one of [1] to [7].
   [15] The method according to [13] or [14], wherein
the disease is the disease specified in [10].
   [16] A method for producing an antisense oligonucleotide for correcting pseudo-exon-type aberrant splicing, including producing a base sequence complementary to a target sequence that includes a branch point in pseudo-exon-type aberrant splicing and consists of 10 to 50 bases.
   [17] The method according to [16], wherein
the antisense oligonucleotide is the antisense oligonucleotide according to any one of [1] to [7].
   [18] The method according to [16] or [17], wherein
the target sequence consists of 10 to 50 successive bases in a region selected from the group consisting of the regions (1) to (40) specified in [7], 10 to 50 successive bases in a region selected from the group consisting of the regions (1) to (23) and (25) to (40) specified in [7], or 10 to 50 successive bases in the region (19) specified in [7].

The present disclosure will be described below in further detail by way of Examples. It is to be noted that these Examples are merely illustrative, and the present disclosure is not limited thereto.

### BRIEF DESCRIPTION OF DRAWINGS

FIG. 1 shows target diseases and target genes of the antisense oligonucleotide of the present disclosure, and mutations that can cause pseudo-exon-type aberrant splicing.

FIG. 2 illustrates design of antisense oligonucleotides (AONs) targeted to a branch point located upstream of a pseudo-exon in the Example.

FIG. 3 shows results obtained in the Example demonstrating the effects of the branch point-targeted antisense oligonucleotides (AONs 1, 2 and 3) on patient-derived myotubes. FIG. 3 shows results of RT-PCR.

FIG. 4 shows results of Western blotting performed in the Example.

FIG. 4 shows glycosylated α-DG from patient-derived myotubes treated with 100 nM of the antisense oligonucleotide 1 (AON 1) or mock-treated. β-DG was used as a loading control. In the graph, data are shown as mean±SD (n=4), and “**” indicates that the statistical significance p<0.01 calculated by t-test using Welch Two Sample t-test.

FIG. 5 shows results of immunofluorescent staining performed in the Example. FIG. 5 shows α-DG glycosylation (clone: IIH6) and myosin heavy chains (MHCs) (clone: MF20) in the patient-derived myotubes after treatment with 100 nM of the antisense oligonucleotide 1 (AON 1) or a mock treatment. The scale bar represents 50 μm. In the graphs, data are shown as mean±SD (n=4), and “**” indicates that the statistical significance p<0.01 calculated by t-test using Welch Two Sample t-test.

FIGS. 6A and 6B illustrate identification of branch points. FIG. 6A schematically shows divergent primers for a lariat intron in a FKTN splicing reporter upstream of a pseudo-exon. P denotes a pseudo-exon, and A denotes a branch point. FIG. 6B illustrates branch points detected by TA cloning and Sanger sequencing, and the main branch point was found to be adenosine located 38 bp from the pseudo-exon (P).

FIG. 7 is a schematic diagram showing a FKTN-mutated reporter and a mutated (#BP_M) reporter, which is the FKTN-mutated reporter with a point mutation. The mutated (#BP_M) reporter has a point mutation (c.647+1977A>T) resulting from substitution of the adenine base as the main branch point with a thymine base. RT-PCR analysis revealed skipping of the pseudo-exon in the mutated (#BP_M) reporter.

FIG. 8 illustrates pseudo-exon-type aberrant splicing in FKTN with a homozygous SVA retrotransposon insertion and design of an antisense oligonucleotide targeted to a branch point. Primers for an aberrant RT-PCR product (black arrows) and primers for a corrected RT-PCR product (gray arrows) are schematically shown.

FIG. 9 shows results obtained in the Example demonstrating the effects of a branch point-targeted antisense oligonucleotide on myotubes derived from patients with a homozygous SVAretrotransposon insertion. FIG. 9 shows RT-PCR analysis after treatment with an antisense oligonucleotide 4 (SVAAON) (30 nM, 100 nM or 300 nM) or a mock treatment. B2-microglobulin was used as a loading control.

FIG. 10 shows results obtained in the Example demonstrating the effects of the branch point-targeted antisense oligonucleotide 4 (SVAAON) on the myotubes derived from the patients with a homozygous SVA retrotransposon insertion. FIG. 10 shows results of RT-qPCR quantification of aberrant splicing and corrected splicing. In the graphs, data are shown as mean±SD (n=4), and “**” indicates that the statistical significance p<0.01 calculated by t-test using Welch Two Sample t-test.

FIG. 11 shows results obtained in the Example demonstrating the effects of the branch point-targeted antisense oligonucleotide on the myotubes derived from the patients with a homozygous SVAretrotransposon insertion. FIG. 11 shows results of Western blotting with respect to glycosylated α-DG treated with 100 nM of the antisense oligonucleotide 4 (SVAAON) or mock-treated. α-DG was used as a loading control. In the graph, data are shown as mean±SD (n=4), and “**” indicates that the statistical significance p<0.01 calculated by t-test using Welch Two Sample t-test.

FIG. 12 shows results of immunofluorescence staining performed in the Example showing the effect of the branch point-targeted antisense oligonucleotide in the myotubes derived from the patients with a homozygous SVAretrotransposon insertion. FIG. 12 shows α-DG glycosylation (clone: IIH6) and myosin heavy chains (MHCs) (clone: MF20) in the patient-derived myotubes after treatment with 100 nM of the antisense oligonucleotide 4 (SVAAON) or a mock treatment. The scale bar represents 50 μm. In the graphs, data are shown as mean±SD (n=4), and “**” indicates that the statistical significance p<0.01 calculated by t-test using Welch Two Sample t-test.

FIG. 13 shows pseudo-exon-type aberrant splicing that may occur in the CTFR (cystic fibrosis) gene.

FIG. 14 shows results obtained in the Example demonstrating the effects of a branch point-targeted antisense oligonucleotide in pseudo-exon-type aberrant splicing that may occur in the CTFR gene. FIG. 14 shows results of RT-PCR analysis after a treatment with an antisense oligonucleotide 5 (BP-AON) (30 nM or 100 nM) or a non-target antisense oligonucleotide (NC) or a mock treatment. The band at 336 bp indicates an aberrant RT-PCR product, and the band at 252 bp indicates a corrected RT-PCR product.

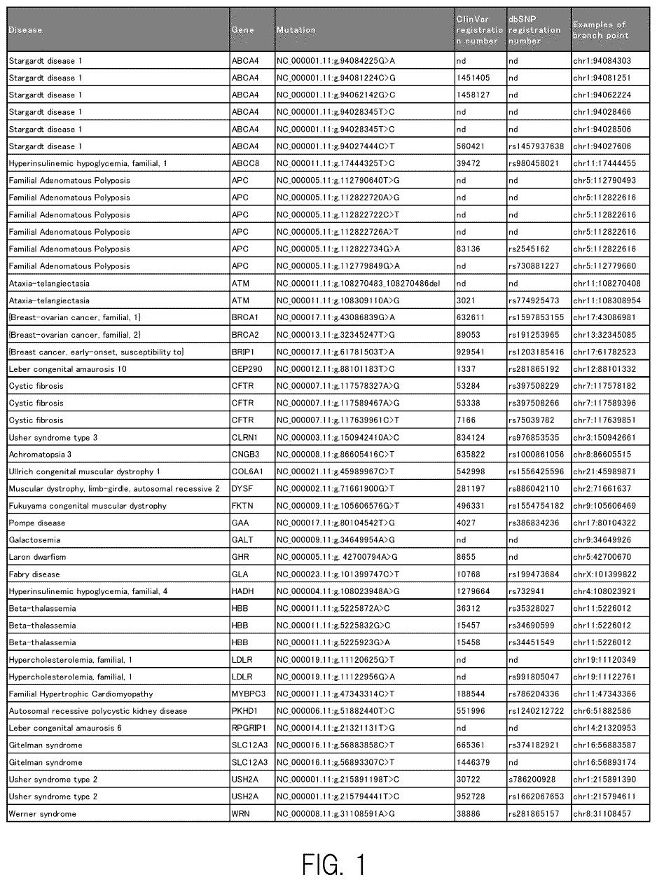

### EXAMPLES

### 1-1. Design of Branch Point-Targeted Antisense Oligonucleotides

The fukutin (FKTN) gene (SEQ ID NO: 14) is the gene responsible for Fukuyama congenital muscular dystrophy. A mutation (c.647+2084G>T (chr9:105606576G>T)) in the FKTN gene is the second most common haplotype of Fukuyama congenital muscular dystrophy. This mutation occurs in intron 6 and causes pseudo-exon-type aberrant splicing creating 64-bp pseudo-exon to be formed between exons 5 and 6.

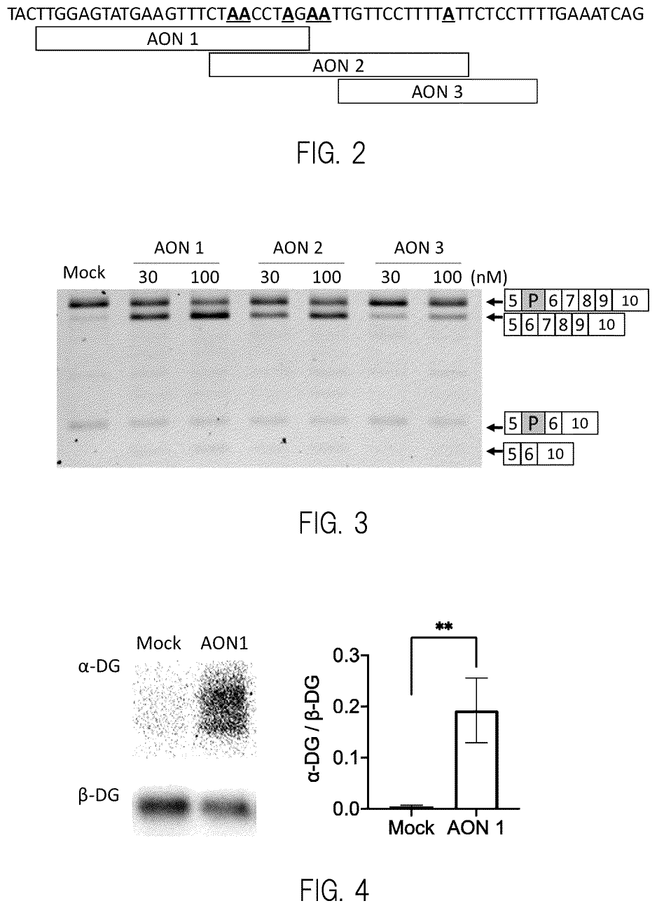

Branch points involved in pseudo-exon-type aberrant splicing caused by the above-described mutation are normally located 120 to 10 bases upstream of the 3′-splice site. The branch points are generally adenosine. In order to determine the optimal target sequence for suppressing this pseudo-exon, three different antisense oligonucleotides were designed (FIG. 2). These antisense oligonucleotides cover all adenosines located within a range from 44 to 18 bases upstream of the pseudo-exon. The antisense oligonucleotides were synthesized using 2′-O-methyl phosphorothioate (Integrated DNA Technologies, Coralville, IA, USA).

Antisense oligonucleotide 1 (AON 1):
(SEQ ID NO: 68)
5′-CUAGGUUAGAAACUUCAUACUCCAA-3′
Antisense oligonucleotide 2 (AON 2):
(SEQ ID NO: 69)
5′-AAUAAAAGGAACAAUUCUAGGUUAG-3′
Antisense oligonucleotide 3 (AON 3):
(SEQ ID NO: 70)
5′-AAAGGAGAAUAAAAGGAACA-3′

The sequence identity of the thus-obtained antisense oligonucleotides 1 to 3 with their target sequences was examined. The antisense oligonucleotide 1 was perfectly complementary to its target sequence (TTGGAGTATGAAGTTTCTAACCTAG: positions 48321 to 48345 of SEQ ID NO: 14), the antisense oligonucleotide 2 was perfectly complementary to its target sequence (CTAACCTAGAATTGTTCCTTTTATT: positions 48337 to 48361 of SEQ ID NO: 14), and the antisense oligonucleotide 3 was perfectly complementary to its target sequence (TGTTCCTTTTATTCTCCTTT: positions 48349 to 48368 of SEQ ID NO: 14).

1-2. The antisense oligonucleotides 1 to 3 restored correct splicing of the mutant allele and increased glycosylated α-dystroglycan expression in myotubes derived from patients with the c.647+2084G>T mutation.

Patient-derived myoblasts were transfected with each of the antisense oligonucleotides 1 to 3. The transfection was performed after culturing the myoblasts in a growth medium until 80% to 100% confluent, and thereafter, the medium was replaced with a differentiation medium to differentiate the myoblasts into myotubes. The antisense oligonucleotide concentration was set to 30 nM or 100 nM, and RNAiMaX Transfection Reagent (Thermo Fisher Scientific) and Opti-MEM (Thermo Fisher Scientific) were used as reagents.

Exons 5 to 10 were subjected to RT-PCR. The results thereof are shown in FIG. 3. P in FIG. 3 denotes “pseudo-exon”. In FIG. 3, the positions of a pseudo-exon-type aberrant splicing product including the pseudo-exon (P), a corrected splicing product, a splicing product including the pseudo-exon (P) and exhibiting skipping of exons 7 to 9, and a splicing product including no pseudo-exon but exhibiting skipping of exons 7 to 9 are shown from the top in this order. As can be seen from FIG. 3, the antisense oligonucleotides 1 to 3 (AONs 1 to 3) suppressed pseudo-exon-type aberrant splicing in a concentration-dependent manner and increased the correctly spliced transcript derived from the mutant allele without causing skipping of other exons. In particular, the antisense oligonucleotides 1 and 2 reduced the pseudo-exon-type aberrant splicing and increased the normal transcript more effectively than the antisense oligonucleotide 3.

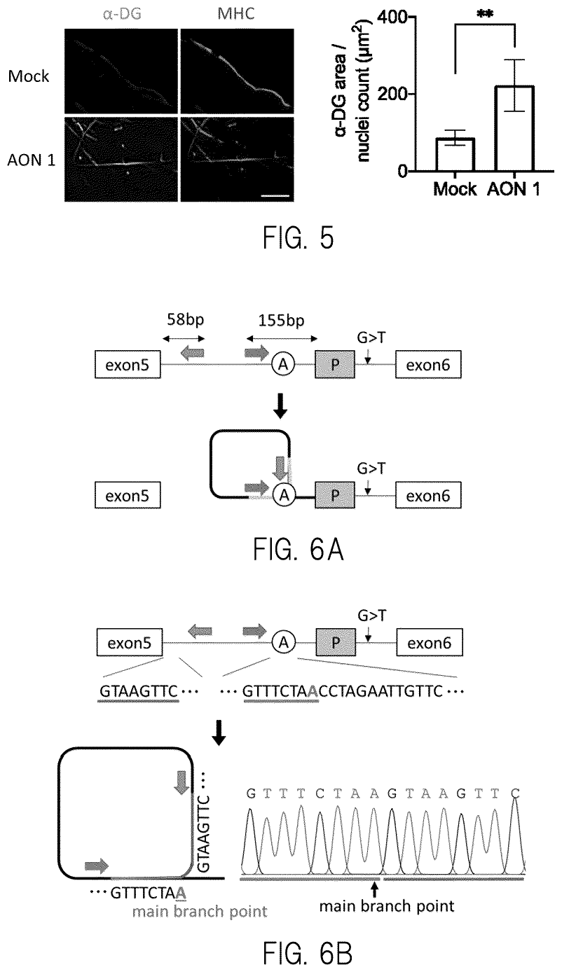

Next, Western blotting and immunofluorescent staining were performed to examine whether the antisense oligonucleotide of the present disclosure restores a decrease in expression of glycosylated α-dystroglycan (α-DG), which is a basic symptom of Fukuyama congenital muscular dystrophy (FCMD). The antisense oligonucleotide 1 was used as the antisense oligonucleotide. The results thereof are shown in FIGS. 4 and 5. FIG. 4 shows the results of the Western blotting, and FIG. 5 shows the results of the immunofluorescence staining. As can be seen from FIGS. 4 and 5, treatment with the antisense oligonucleotide 1 increased the expression of glycosylated α-DG as compared with the case where the antisense oligonucleotide 1 was not added. In particular, a prominent increase in the expression was observed in the immunofluorescent staining. These results demonstrate that the antisense oligonucleotide of the present disclosure is capable of restoring basic FKTN expression in FCMD patients.

1-3. Identification of Branch Points from Lariat Introns

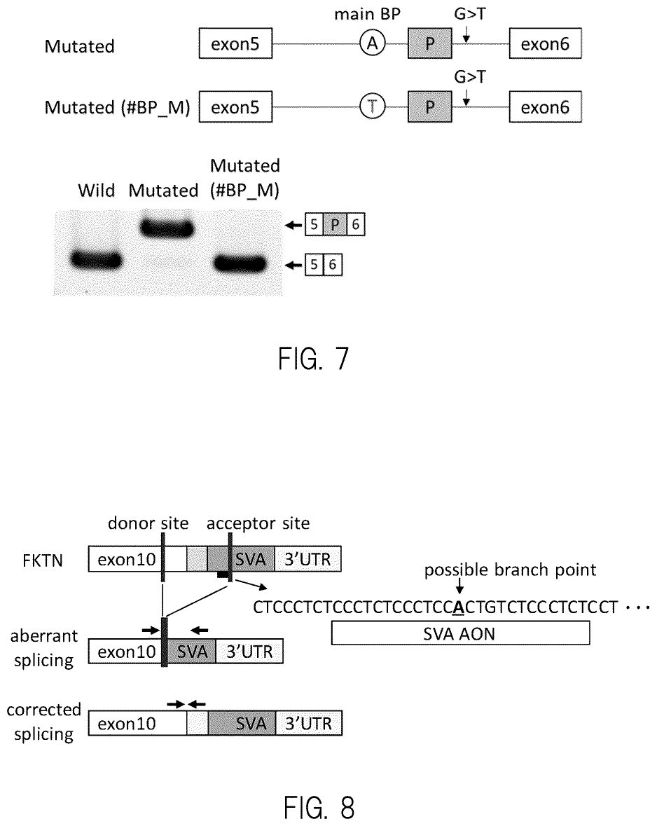

Whether the antisense oligonucleotide 1 regulated splicing by actually inhibiting the branch point was examined. In order to identify the branch point used for the pseudo-exon in FKTN-mRNA, a lariat intron in a FKTN splicing reporter was subjected to RT-PCR analysis. As shown in FIG. 6A, divergent primers for upstream of the pseudo-exon were designed in order to detect lariat introns across branch points, and RT-PCR products with the predicted size were detected. In 76% of RT-PCR products, adenosine located 38 bp upstream from the pseudo-exon (c.647+1977A, 48340th base in SEQ ID NO: 14) was used as the branch point, as shown in FIG. 6B. In order to examine whether pseudo-exon skipping is caused by inhibiting the branch point, a mutated (#BP_M) reporter having a point mutation (c.647+1977A>T) was produced (FIG. 7). HEK293 was transfected with a wild-type reporter, a mutated reporter, and the mutated (#BP_M) reporter, and the occurrence of pseudo-exon skipping in FKTN-mRNA was examined. As can be seen from FIG. 7, FKTN-mRNA derived from the mutated reporter included the pseudo-exon. On the other hand, FKTN-mRNA derived from the mutated (#BP_M) reporter did not include the pseudo-exon, as with FKTN-mRNAfrom the wild-type reporter.

Since the antisense oligonucleotides 1 and 2 include the branch point (c.647+1977A) used for the pseudo-exon in FKTN-mRNA, it can be said that these results demonstrate the antisense oligonucleotides 1 and 2 skip the pseudo-exon.

Reference Example: Design of an Antisense Oligonucleotide Targeted to a Branch Point in FCMD with a Homozygous SVA Retrotransposon Insertion

In order to evaluate whether branch point-targeted antisense oligonucleotides are also effective for mutations other than pseudo-exon mutations, an antisense oligonucleotide was designed for a SINE-VNTR-Alu (SVA) retrotransposon insertion mutation in FCMD. The SVAretrotransposon insertion occurs in the 3′-untranslated region (UTR) of the FKTN gene, and results in pseudo-exon-type aberrant splicing due to SVA exon trapping. There was only one adenosine located within a range from 18 to 44 bases upstream of the acceptor site. Thus, this adenosine was determined as the branch point, and from a region including this branch point (CTCCCTCTCCCTCTCCCTCCACTGTCTCCCTCTCCT: SEQ ID NO: 71), a target sequence (CCCTCTCCCTCCACTGTCTCCCTCT: SEQ ID NO: 72) was determined. Then, an antisense oligonucleotide 4 targeted to this branch point was designed (FIG. 8). The antisense oligonucleotide 4 was perfectly complementary to the target sequence (CCCTCTCCCTCCACTGTCTCCCTCT: SEQ ID NO: 72).

Antisense Oligonucleotide 4 (SVAAON):
(SEQ ID NO: 73)
5′-AGAGGGAGACAGUGGAGGGAGAGGG-3′

2. The Antisense Oligonucleotide 4 Rectifies Aberrant Splicing and α-Dystroglycan Glycosylation in Myotubes Derived from Patients with a Homozygous SVA Retrotransposon Insertion

Patient-derived myoblasts with a homozygous SVA retrotransposon insertion were transfected with the antisense oligonucleotide 4. The transfection was performed in the same manner as described in the item 1-2 above, except that the antisense oligonucleotide 4 was used and the concentration of the antisense oligonucleotide 4 was set to 30 nM, 100 nM, or 300 nM.

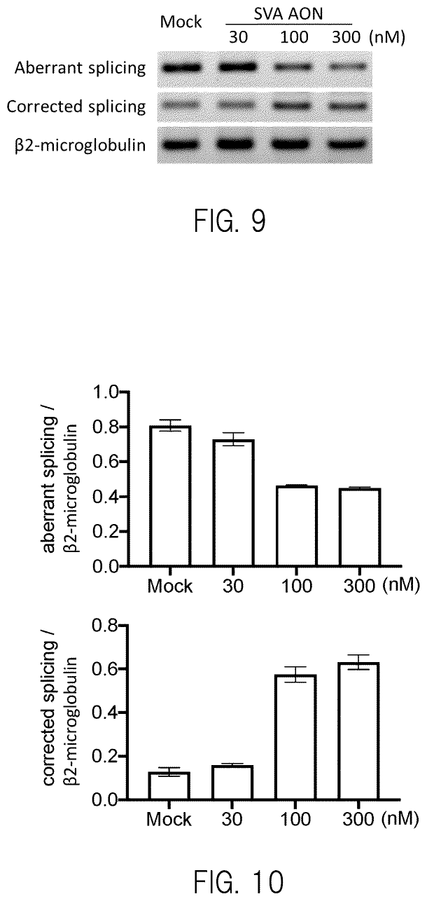

The results thereof are shown in FIG. 9. As can be seen from FIG. 9, the antisense oligonucleotide 4 suppressed pseudo-exon-type aberrant splicing in a dose-dependent manner and increased the number of correctly spliced transcripts derived from the mutant allele. RT-PCR was also performed to examine the effect of the antisense oligonucleotide 4. The results thereof are shown in FIG. 10. The antisense oligonucleotide 4 reduced the pseudo-exon-type aberrant splicing in a concentration-dependent manner, and reduced the pseudo-exon-type aberrant splicing to 56% at 300 nM. Also, the antisense oligonucleotide 4 increased a normal splicing product in a dose-dependent manner, and increased the number of correctly spliced products to 493% at 300 nM.

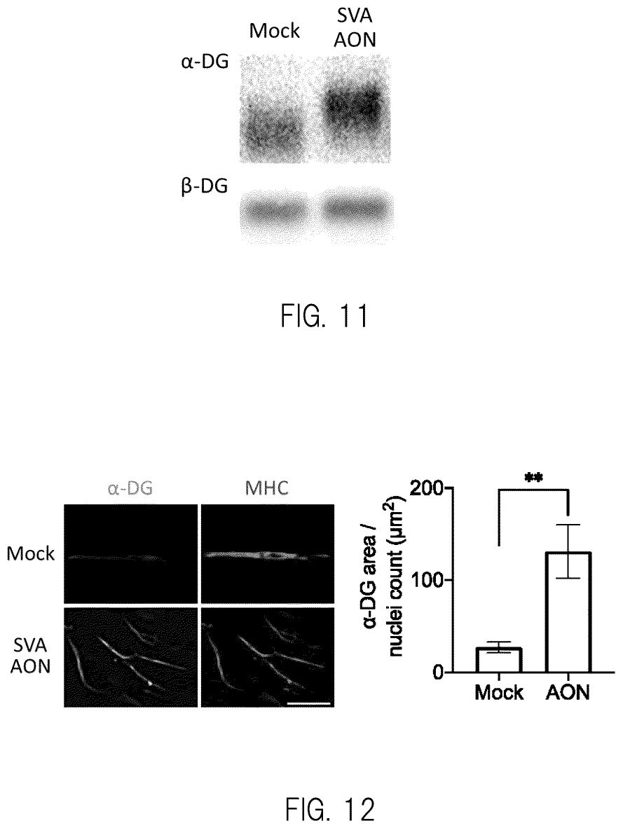

Next, Western blotting and immunofluorescent staining were performed to examine whether the antisense oligonucleotide of the present disclosure restores a decrease in expression of glycosylated α-dystroglycan (α-DG), which is a basic symptom of FCMD. The results thereof are shown in FIGS. 11 and 12. The Western blotting of glycosylated α-DG showed a significant shift toward a higher molecular weight as compared with the case without the treatment with the antisense oligonucleotide 4 (FIG. 11). The immunofluorescent staining showed a marked increase of glycosylated α-DG (FIG. 12). These results demonstrate that the branch point-targeted antisense oligonucleotide 4 is capable of restoring basic FKTN expression in FCMD patients.

3-1. Design of an Antisense Oligonucleotide Targeted to a Branch Point in Pseudo-Exon-Type Aberrant Splicing that May be Caused in the CTFR Gene

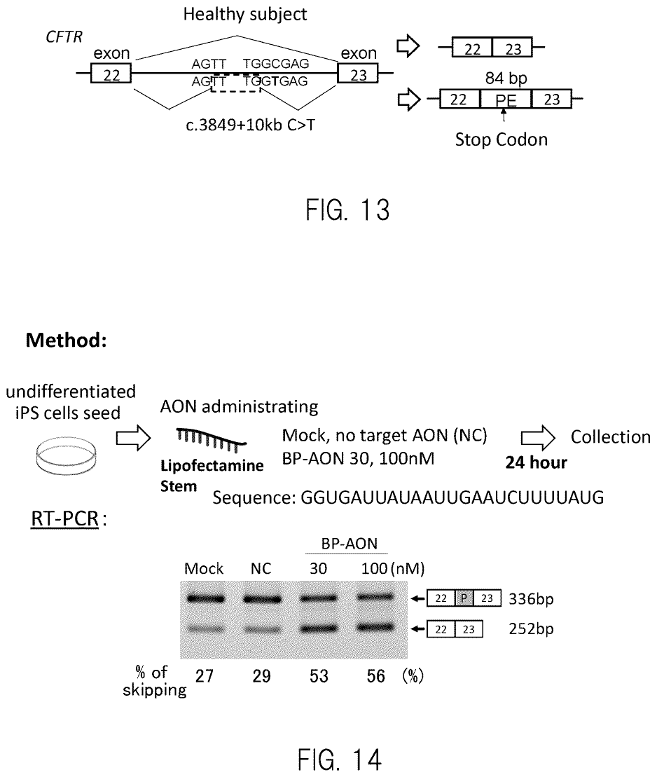

The CFTR gene (SEQ ID NO: 9) is a gene responsible for cystic fibrosis. A mutation (c.3849+12191C>T (chr7:117639961C>T)) in the CFTR gene occurs in intron 22, and causes 84-bp pseudo-exon to be formed between exons 22 and 23 (FIG. 13). An adenosine (chr7:117639851) located 10 bases upstream from the above-described mutation (c.3849+12191C>T (chr7:117639961C>T)) causing the pseudo-exon was determined as the branch point, and an antisense oligonucleotide 5 targeted to this branch point was designed and synthesized using 2′-O-methyl phosphorothioate (Integrated DNA Technologies, Coralville, IA, USA).

Antisense oligonucleotide 5 (BP-AON):
(SEQ ID NO: 74)
5′-GGUGAUUAUAAUUGAAUCUUUUAUG-3′

3-2. The Antisense Oligonucleotide 5 (BP-AON) Rectifies Aberrant Splicing in Undifferentiated iPS Cells Derived from Patients with the c.3849+12191C>T Mutation

First, undifferentiated iPS cells generated from patients with the c.3849+12191C>T mutation were prepared. Then, the undifferentiated iPS cells were cultured in a growth medium until 80% to 100% confluent, and then transfected with the antisense oligonucleotide 5. The cells were collected 24 hours later. The concentration of the antisense oligonucleotide 5 was set to 30 nM or 100 nM, and Lipofectamine Stem Transfection Reagent (Thermo Fisher Scientific) was used as a reagent (FIG. 14). Then, exons 22 to 23 were subjected to RT-PCR. As a control, the transfection was performed in the same manner using, instead of the antisense oligonucleotide 5, an antisense oligonucleotide (NC) for a non-target nucleic acid sequence.

The results thereof are shown in FIG. 14. As can be seen from FIG. 14, the antisense oligonucleotide 5 suppressed pseudo-exon-type aberrant splicing and increased a correctly spliced transcript. In addition, although the percentage of normal splicing product was about 27% in the mock control, the antisense oligonucleotide 5 could restore the number of correctly splicing products to 56%.

1. An antisense oligonucleotide for correcting pseudo-exon-type aberrant splicing, having a base sequence capable of binding to a sequence that includes a branch point in pseudo-exon-type aberrant splicing.

2. The antisense oligonucleotide according to claim 1, wherein
the pseudo-exon-type aberrant splicing is aberrant splicing caused by recognition of a non-coding region as a pseudo-exon, and
the branch point is an adenine base.

3. The antisense oligonucleotide according to claim 2 for inducing and/or promoting skipping of the pseudo-exon by binding to the sequence including the branch point.

4. The antisense oligonucleotide according to claim 1, wherein
the branch point is an adenine base located upstream of a 3′-splice site of an intron.

5. The antisense oligonucleotide according to claim 1, wherein
the branch point is an adenine base located in a region from 10 to 120 bases upstream of a 3′-splice site of an intron.

6. The antisense oligonucleotide according to claim 1, consisting of 10 to 50 bases.

7. An antisense oligonucleotide comprising a base sequence capable of binding to a target sequence consisting of 10 to 50 successive bases in a region selected from the group consisting of the following regions (1) to (40),
wherein the base sequence includes a branch point in pseudo-exon-type aberrant splicing:
(1) a region that is in an ABCA4 gene and corresponds to a region from 94084339th to 94084229th bases (SEQ ID NO: 28) in a base sequence of human chromosome 1 in a GRCh38/hg38 human reference genome list;
(2) a region that is in the ABCA4 gene and corresponds to a region from 94081341st to 94081231st bases (SEQ ID NO: 29) in the base sequence of human chromosome 1 in the GRCh38/hg38 human reference genome list;
(3) a region that is in the ABCA4 gene and corresponds to a region from 94062301st to 94062191st bases (SEQ ID NO: 30) in the base sequence of human chromosome 1 in the GRCh38/hg38 human reference genome list;
(4) a region that is in the ABCA4 gene and corresponds to a region from 94028532nd to 94028422nd bases (SEQ ID NO: 31) in the base sequence of human chromosome 1 in the GRCh38/hg38 human reference genome list;
(5) a region that is in the ABCA4 gene and corresponds to a region from 94028576th to 94028466th bases (SEQ ID NO: 32) in the base sequence of human chromosome 1 in the GRCh38/hg38 human reference genome list;
(6) a region that is in the ABCA4 gene and corresponds to a region from 94027674th to 94027564th bases (SEQ ID NO: 33) in the base sequence of human chromosome 1 in the GRCh38/hg38 human reference genome list;
(7) a region that is in an ABCC8 gene and corresponds to a region from 17444521st to 17444411th bases (SEQ ID NO: 34) in a base sequence of human chromosome 11 in the GRCh38/hg38 human reference genome list;
(8) a region that is in an APC gene and corresponds to a region from 112790393rd to 112790503rd bases (SEQ ID NO: 35) in a base sequence of human chromosome 5 in the GRCh38/hg38 human reference genome list;
(9) a region that is in the APC gene and corresponds to a region from 112822518th to 112822628th bases (SEQ ID NO: 36) in the base sequence of human chromosome 5 in the GRCh38/hg38 human reference genome list;
(10) a region that is in the APC gene and corresponds to a region from 112779563rd to 112779673rd bases (SEQ ID NO: 37) in the base sequence of human chromosome 5 in the GRCh38/hg38 human reference genome list;
(11) a region that is in an ATM gene and corresponds to a region from 108270351st to 108270461st bases (SEQ ID NO: 38) in the base sequence of human chromosome 11 in the GRCh38/hg38 human reference genome list;
(12) a region that is in the ATM gene and corresponds to a region from 108308849th to 108308959th bases (SEQ ID NO: 39) in the base sequence of human chromosome 11 in the GRCh38/hg38 human reference genome list;
(13) a region that is in a BRCA1 gene and corresponds to a region from 43087074th to 43086964th bases (SEQ ID NO: 40) in a base sequence of human chromosome 17 in the GRCh38/hg38 human reference genome list;
(14) a region that is in a BRCA2 gene and corresponds to a region from 32345028th to 32345138th bases (SEQ ID NO: 41) in a base sequence of human chromosome 13 in the GRCh38/hg38 human reference genome list;
(15) a region that is in a BRIP1 gene and corresponds to a region from 61/782,587th to 61/782,477th bases (SEQ ID NO: 42) in the base sequence of human chromosome 17 in the GRCh38/hg38 human reference genome list;
(16) a region that is in a CEP290 gene and corresponds to a region from 88101435th to 88101325th bases (SEQ ID NO: 43) in a base sequence of human chromosome 12 in the GRCh38/hg38 human reference genome list;
(17) a region that is in a CFTR gene and corresponds to a region from 117578099th to 117578209th bases (SEQ ID NO: 44) in a base sequence of human chromosome 7 in the GRCh38/hg38 human reference genome list;
(18) a region that is in the CFTR gene and corresponds to a region from 117589298th to 117589408th bases (SEQ ID NO: 45) in the base sequence of human chromosome 7 in the GRCh38/hg38 human reference genome list;
(19) a region that is in the CFTR gene and corresponds to a region from 117639756th to 117639866th bases (SEQ ID NO: 46) in the base sequence of human chromosome 7 in the GRCh38/hg38 human reference genome list;
(20) a region that is in a CLRN1 gene and corresponds to a region from 150942759th to 150942649th bases (SEQ ID NO: 47) in a base sequence of human chromosome 3 in the GRCh38/hg38 human reference genome list;
(21) a region that is in a CNGB3 gene and corresponds to a region from 86605573rd to 86605463rd bases (SEQ ID NO: 48) in a base sequence of human chromosome 8 in the GRCh38/hg38 human reference genome list;
(22) a region that is in a COL6A1 gene and corresponds to a region from 45989774th to 45989884th bases (SEQ ID NO: 49) in a base sequence of human chromosome 21 in the GRCh38/hg38 human reference genome list;
(23) a region that is in a DYSF gene and corresponds to a region from 71661602nd to 71661712th bases (SEQ ID NO: 50) in a base sequence of human chromosome 2 in the GRCh38/hg38 human reference genome list;
(24) a region that is in a FKTN gene and corresponds to a region from 105606387th to 105606497th bases (SEQ ID NO: 51) in a base sequence of human chromosome 9 in the GRCh38/hg38 human reference genome list;
(25) a region that is in a GAA gene and corresponds to a region from 80104281st to 80104391st bases (SEQ ID NO: 52) in the base sequence of human chromosome 17 in the GRCh38/hg38 human reference genome list;
(26) a region that is in a GALT gene and corresponds to a region from 34649835th to 34649945th bases (SEQ ID NO: 53) in the base sequence of human chromosome 9 in the GRCh38/hg38 human reference genome list;
(27) a region that is in a GHR gene and corresponds to a region from 42700567th to 42700677th bases (SEQ ID NO: 54) in the base sequence of human chromosome 5 in the GRCh38/hg38 human reference genome list;
(28) a region that is in a GLA gene and corresponds to a region from 101399920th to 101399810th bases (SEQ ID NO: 55) in a base sequence of human chromosome X in the GRCh38/hg38 human reference genome list;
(29) a region that is in an HADH gene and corresponds to a region from 108023829th to 108023939th bases (SEQ ID NO: 56) in a base sequence of human chromosome 4 in the GRCh38/hg38 human reference genome list;
(30) a region that is in an HBB gene and corresponds to a region from 5226117th to 5226007th bases (SEQ ID NO: 57) in the base sequence of human chromosome 11 in the GRCh38/hg38 human reference genome list;
(31) a region that is in an LDLR gene and corresponds to a region from 11120250th to 11120360th bases (SEQ ID NO: 58) in a base sequence of human chromosome 19 in the GRCh38/hg38 human reference genome list;
(32) a region that is in the LDLR gene and corresponds to a region from 11122702nd to 11122812th bases (SEQ ID NO: 59) in the base sequence of human chromosome 19 in the GRCh38/hg38 human reference genome list;
(33) a region that is in an MYBPC3 gene and corresponds to a region from 47343432nd to 47343322nd bases (SEQ ID NO: 60) in the base sequence of human chromosome 11 in the GRCh38/hg38 human reference genome list;
(34) a region that is in a PKHD1 gene and corresponds to a region from 51882676th to 51882566th bases (SEQ ID NO: 61) in a base sequence of human chromosome 6 in the GRCh38/hg38 human reference genome list;
(35) a region that is in an RPGRIP1 gene and corresponds to a region from 21320889th to 21320999th bases (SEQ ID NO: 62) in a base sequence of human chromosome 14 in the GRCh38/hg38 human reference genome list;
(36) a region that is in an SLC12A3 gene and corresponds to a region from 56883499th to 56883609th bases (SEQ ID NO: 63) in a base sequence of human chromosome 16 in the GRCh38/hg38 human reference genome list;
(37) a region that is in the SLC12A3 gene and corresponds to a region from 56893096th to 56893206th bases (SEQ ID NO: 64) in the base sequence of human chromosome 16 in the GRCh38/hg38 human reference genome list;
(38) a region that is in a USH2A gene and corresponds to a region from 215891470th to 215891360th bases (SEQ ID NO: 65) in the base sequence of human chromosome 1 in the GRCh38/hg38 human reference genome list;
(39) a region that is in the USH2A gene and corresponds to a region from 215794716th to 215794606th bases (SEQ ID NO: 66) in the base sequence of human chromosome 1 in the GRCh38/hg38 human reference genome list; and
(40) a region that is in a WRN gene and corresponds to a region from 31108365th to 31108475th bases (SEQ ID NO: 67) in the base sequence of human chromosome 8 in the GRCh38/hg38 human reference genome list.

8. A pharmaceutical composition for correcting pseudo-exon-type aberrant splicing, comprising the antisense oligonucleotide according to claim 1 as an active ingredient.

9. A pharmaceutical composition for preventing or treating the development of a disease caused by pseudo-exon-type aberrant splicing, comprising the antisense oligonucleotide according to claim 1 as an active ingredient.

10. The pharmaceutical composition according to claim 9, wherein the disease is at least one selected from the group consisting of: Stargardt disease; hyperinsulinemic hypoglycemia, familial; familial adenomatous polyposis; ataxia-telangiectasia; breast-ovarian cancer, familial; breast cancer, early-onset, susceptibility to; Leber congenital amaurosis; cystic fibrosis; Usher syndrome; achromatopsia; Ullrich congenital muscular dystrophy; muscular dystrophy, limb-girdle, autosomal recessive; Fukuyama congenital muscular dystrophy; Pompe disease; galactosemia; Laron dwarfism; Fabry disease; beta-thalassemia; hypercholesterolemia, familial; familial hypertrophic cardiomyopathy; autosomal recessive polycystic kidney disease; Gitelman syndrome; and Werner syndrome.

11. A method for correcting pseudo-exon-type aberrant splicing using an antisense oligonucleotide having a base sequence capable of binding to a sequence that includes a branch point in pseudo-exon-type aberrant splicing,
wherein the antisense oligonucleotide is the antisense oligonucleotide according to claim 1.

12. A method for treating a disease caused by pseudo-exon-type aberrant splicing, comprising administering to a subject an antisense oligonucleotide having a base sequence capable of binding to a sequence that includes a branch point in pseudo-exon-type aberrant splicing,
wherein the antisense oligonucleotide is the antisense oligonucleotide according to claim 1.

13. A method for producing an antisense oligonucleotide for correcting pseudo-exon-type aberrant splicing, comprising producing a base sequence complementary to a target sequence that includes a branch point in pseudo-exon-type aberrant splicing and consists of 10 to 50 bases.
